(19) United States
(12) Patent Application Publication
(10) Pub. No.: US 2026/0098648 A1
(43) Pub. Date: Apr. 9, 2026

(54) INDOOR AIR POLLUTION-FREE CLEANROOM SYSTEM ACHIEVING ARTIFICIAL INTELLIGENCE GREEN AND HEALTH BUILDING STANDARDS

(71) Applicant: Microjet Technology Co., Ltd., Hsinchu (TW)

(72) Inventors: Hao-Jan Mou, Hsinchu (TW); Chin-Chuan Wu, Hsinchu (TW); Chi-Feng Huang, Hsinchu (TW)

(73) Assignee: Microjet Technology Co., Ltd., Hsinchu (TW)

(21) Appl. No.: 19/306,447

(22) Filed: Aug. 21, 2025

(30) Foreign Application Priority Data
Oct. 4, 2024 (TW) 113137940

(51) Int. Cl. F24F 3/167; A61L 9/20; A61L 9/22; B01D 46/00; F24F 8/108; F24F 8/15; F24F 8/22; F24F 8/30; F24F 11/58; F24F 11/64; F24F 110/65

(52) U.S. Cl. CPC F24F 3/167; A61L 9/205; A61L 9/22; B01D 46/0028; B01D 46/0036; F24F 8/108; F24F 8/15; F24F 8/22; F24F 8/30; F24F 11/58; F24F 11/64; A61L 2209/111; A61L 2209/12; A61L 2209/14; A61L 2209/16; B01D 2273/30; B01D 2279/51; B01D 2279/65; F24F 2110/65

(57) ABSTRACT

An indoor air pollution-free cleanroom system which achieves artificial intelligence green and health building standards is disclosed. Based on an indoor air pollution detection and purification system combined with an efficient building design structure, a renewable energy system, a water resources management system, a circular economy and resource recycling, the requirements of high comfort and low energy consumption of the green building standards (LEED standards) and the healthy building standards (WELL standards) are achieved, the new artificial intelligence green and healthy building standards is achieved, and the advantages of controlling indoor air pollution, water pollution, noise pollution and carbon emission to be zero, extremely saving energy, improving the health and comfort of the living environment are achieved.

### CROSS-REFERENCE TO RELATED APPLICATION

This application claims priority to Taiwan Patent Application No. 113137940, filed on Oct. 4, 2024. The entire contents of the above-mentioned patent application are incorporated herein by reference for all purposes.

### FIELD OF THE INVENTION

The present disclosure relates to an indoor air pollution-free cleanroom system that achieves artificial intelligence green and healthy building standards, and more particularly to an indoor air pollution-free cleanroom system combining a net zero carbon emission goal with a high-efficiency air purification technology to achieve the new artificial intelligence green and healthy building standards, thereby controlling the indoor air pollution to be completely clean, the water pollution to zero, the noise pollution to zero and the net carbon emissions to be zero, maximizing the energy saving, and improving the health and comfort of the living environment.

### BACKGROUND OF THE INVENTION

With the acceleration of urbanization and the rising demands for environmental quality, the indoor air quality and the building energy consumption are gaining increasing attention. The net-zero carbon emissions and the indoor air quality have become crucial topics. The existing building designs and the air purification technologies cannot simultaneously meet both requirements, particularly in terms of intelligent regulation and addressing the complex pollution sources. In order to address these issues, the present disclosure introduces an intelligently controlled networked system to achieve a comprehensive solution for the net-zero carbon emissions and the zero air pollution, so as to meet the high standards for the indoor air quality. This system can be customized to fit different buildings and indoor air quality requirements, not only for meeting the low energy consumption and the high comfort requirements of the LEED (Leadership in Energy and Environmental Design) standard and the WELL building standard, but also for meeting the new artificial intelligence green and healthy building standards. Thereby, it allows to control the indoor air pollution to be completely clean, the water pollution to zero, the noise pollution to zero and the net carbon emissions to be zero, maximize the energy saving, and improve the health and comfort of the living environment.

### SUMMARY OF THE INVENTION

One object of the present disclosure is to provide an indoor air pollution-free cleanroom system that achieves artificial intelligence green and healthy building standards. An intelligently controlled indoor air pollution detection and purification system and an advanced indoor and outdoor air pollution treatment device are integrated with an efficient building design structure, a renewable energy system, a water resource management system, a circular economy and resource recycling, and combined with a net zero carbon emission goal and an efficient air purification technology, and it allows to achieve low energy consumption and high comfort requirements in green building standards (LEED standards) and healthy building standards (WELL standards), and achieve new artificial intelligence green and healthy building standards, thereby controlling the indoor air pollution to be completely clean, the water pollution to zero, the noise pollution to zero and the net carbon emissions to be zero, maximizing the energy saving, and improving the health and comfort of the living environment.

In accordance with an aspect of the present disclosure, an indoor air pollution-free cleanroom system achieving artificial intelligence green and healthy building standards is provided, and includes an indoor air pollution detection and purification system, a high-efficiency building design structure, a renewable energy system, a water resource management system, and a circular economy and resource recycling. The indoor air pollution detection and purification system includes a plurality of gas detectors, at least one indoor air pollution treatment device, at least one networked cloud computing service device and at least one storage and data processing device, wherein the plurality of gas detectors comprehensively monitor indoor air pollution, the indoor air pollution treatment device filters and purifies the indoor air pollution, an intelligent networking system of the networked cloud computing service device and an artificial intelligence generated content (AIGC) model are used to perform dynamic monitoring and adjustment analysis and a control instruction is issued based on analysis results, to adjust an operating mode of the indoor air pollution treatment device, whereby the indoor air purification effect is achieved, the pollution-free indoor air quality is achieved and the cleanliness meets the cleanroom level. The high-efficiency building design structure includes a passive design structure and an airtight design structure, wherein the passive design structure includes a natural ventilation structure (not shown), a natural lighting structure (not shown) and a thermal insulation structure (not shown) for reducing the need for air conditioning and artificial lighting. The airtight design structure is used to improve a building airtightness, and further combined with an intelligent ventilation system to ensure air circulation and avoid the accumulation of pollutants. The renewable energy system includes a solar photovoltaic system, a wind energy system, a geothermal energy system and an energy storage system for providing electricity to a building and storing excess electricity to meet peak electricity demand. The water resource management system includes a rainwater collection system and a high-efficiency water-saving device, wherein the rainwater collection system is used to recycle rainwater, wherein the high-efficiency water-saving device includes a low-flow toilet and an intelligent sprinkler system configured to use rainwater recycled by the rainwater collection system to save water. The circular economy and resource recycling includes a building material recycling and a construction resource recycling, wherein the building material recycling is configured to recycle building demolition or renovation materials, and the construction resource recycling is configured to promote an efficient use of resources within a building life cycle.

### BRIEF DESCRIPTION OF THE DRAWINGS

FIG. 1A is a schematic view illustrating an indoor air pollution-free cleanroom system achieving artificial intelligence green and healthy building standards according to an embodiment of the present disclosure;

FIG. 1B is a schematic view illustrating the indoor air pollution detection and purification system according to the embodiment of the present disclosure;

FIG. 1C is a schematic view illustrating the indoor air pollution treatment device according to the embodiment of the present disclosure;

FIG. 1D is a schematic view illustrating the indoor air pollution-free cleanroom system implemented in an indoor field according to the embodiment of the present disclosure;

FIG. 2A is a schematic view illustrating the gas exchange device of the indoor air pollution treatment device according to the embodiment of the present disclosure;

FIG. 2B is a schematic view illustrating the purifier of the indoor air pollution treatment device according to the embodiment of the present disclosure;

FIG. 2C is a schematic cross-sectional view illustrating the purifier of the indoor air pollution treatment device in FIG. 1D and FIG. 2B of the present disclosure;

FIG. 2D is a schematic view illustrating the fan filter unit (FFU) of the indoor air pollution treatment device according to the embodiment of the present disclosure;

FIG. 2E is a schematic cross-sectional view illustrating the humidity control device of the indoor air pollution treatment device in FIG. 1D of the present disclosure;

FIG. 2F is a schematic cross-sectional view illustrating the mobile vacuum cleaner of the indoor air pollution treatment device in FIG. 1D of the present disclosure;

FIG. 2G is a flow chart illustrating the gas exchange device comparing the carbon dioxide (CO2) pressure difference between the indoor field and the outdoor field through the networked cloud computing service device online to control a positive pressure air intake according to the embodiment of the present disclosure;

FIG. 2H is a schematic cross-sectional view illustrating the filter component of the indoor air pollution processing device according to the embodiment of the present disclosure;

FIG. 3A is a schematic perspective view illustrating the gas detector according to the embodiment of the present disclosure;

FIG. 3B is a schematic perspective view illustrating the gas detector according to the embodiment of the present disclosure and taken from another perspective;

FIG. 3C is a schematic perspective view illustrating the gas detection module installed inside the gas detector according to the embodiment of the present disclosure;

FIG. 4A is a schematic perspective view (1) illustrating the gas detection main part according to the embodiment of the present disclosure;

FIG. 4B is a schematic perspective view (2) illustrating the gas detection main part according to the embodiment of the present disclosure;

FIG. 4C is an exploded view illustrating the gas detection device according to the embodiment of the present disclosure;

FIG. 5A is a schematic perspective view (1) illustrating the base according to the embodiment of the present disclosure;

FIG. 5B is a schematic perspective view (2) illustrating the base according to the embodiment of the present disclosure;

FIG. 6 is a schematic view (3) illustrating the base according to the embodiment of the present disclosure;

FIG. 7A is a schematic exploded view illustrating the combination of the piezoelectric actuator and the base according to the embodiment of the present disclosure;

FIG. 7B is a schematic perspective view illustrating the combination of the piezoelectric actuator and the base according to the embodiment of the present disclosure;

FIG. 8A is a schematic exploded view (1) illustrating the piezoelectric actuator according to the embodiment of the present disclosure;

FIG. 8B is a schematic exploded view (2) illustrating the piezoelectric actuator according to the embodiment of the present disclosure;

FIG. 9A is a schematic cross-sectional view (1) illustrating an action of the piezoelectric actuator according to the embodiment of the present disclosure;

FIG. 9B is a schematic cross-sectional view (2) illustrating an action of the piezoelectric actuator according to the embodiment of the present disclosure;

FIG. 9C is a schematic cross-sectional view (3) illustrating an action of the piezoelectric actuator according to the embodiment of the present disclosure;

FIG. 10A is a schematic cross-sectional view (1) illustrating the gas detection main part according to the embodiment of the present disclosure;

FIG. 10B is a schematic cross-sectional view (2) illustrating the gas detection main part according to the embodiment of the present disclosure;

FIG. 10C is a schematic cross-sectional view (3) illustrating the gas detection main part according to the embodiment of the present disclosure;

FIG. 11 is a schematic diagram illustrating the communication transmission of the gas detector according to the embodiment of the present disclosure;

FIG. 12A is a schematic diagram illustrating the networked cloud computing service device according to the embodiment of the present disclosure; and FIG. 12B is a schematic diagram of the architecture of the artificial intelligence generated content (AIGC) model of the networked cloud computing service device according to the embodiment of the present disclosure.

### DETAILED DESCRIPTION OF THE PREFERRED EMBODIMENT

The present disclosure will now be described more specifically with reference to the following embodiments. It is to be noted that the following descriptions of preferred embodiments of this disclosure are presented herein for purpose of illustration and description only. It is not intended to be exhaustive or to be limited to the precise form disclosed.

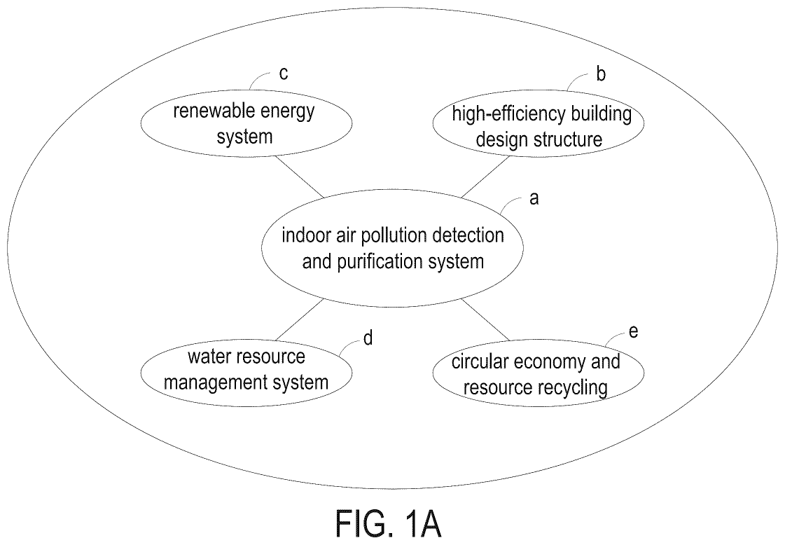

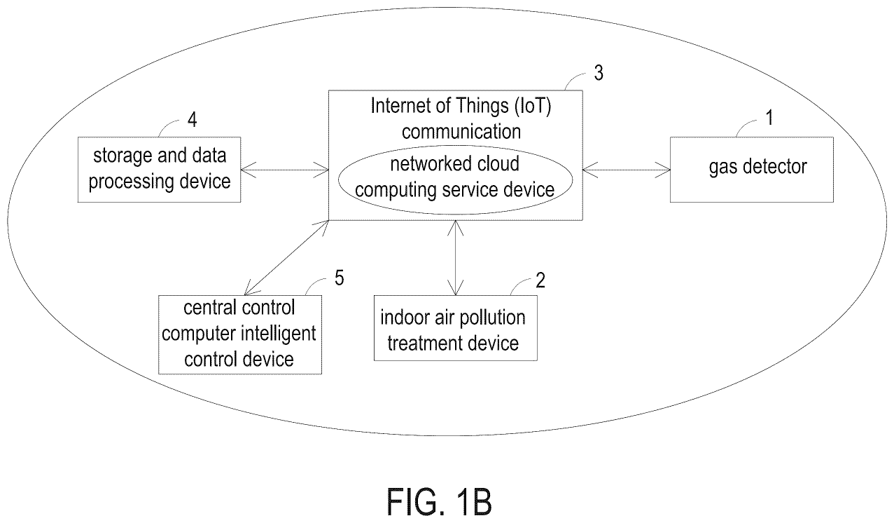

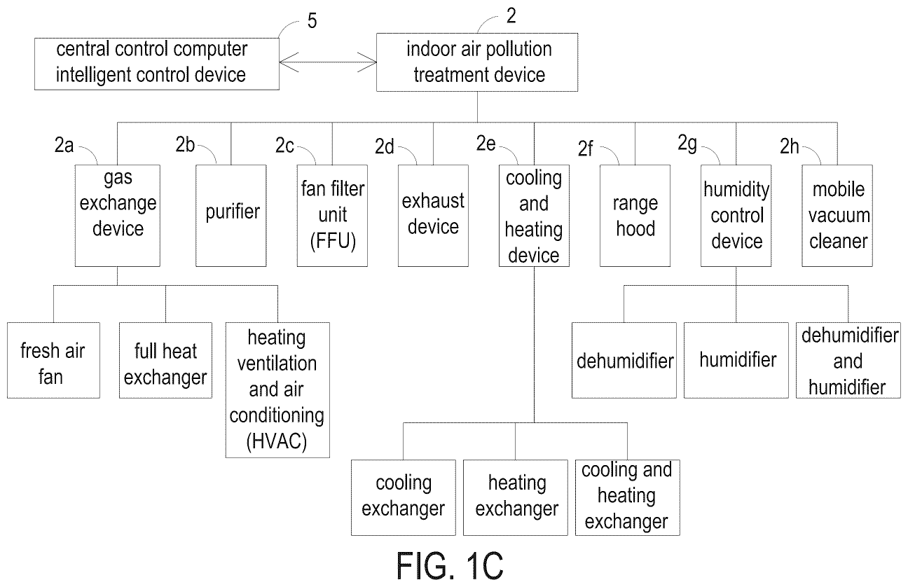

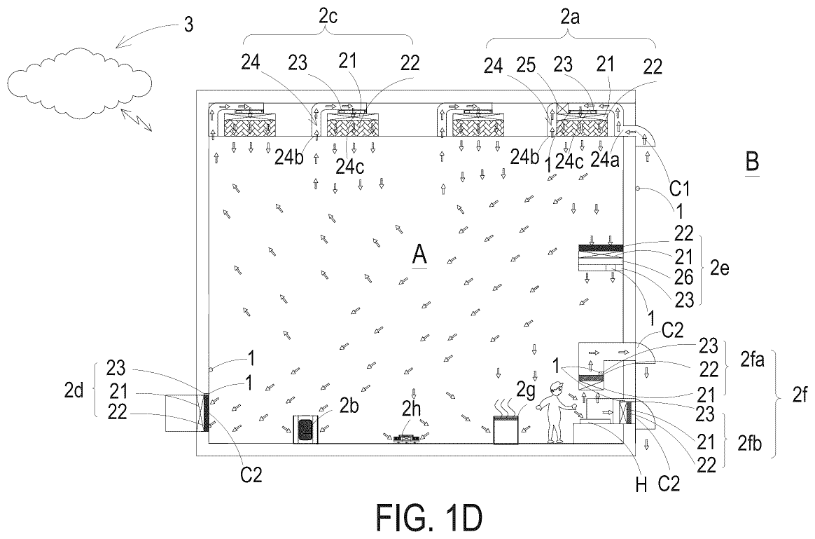

As shown in FIG. 1A, the present disclosure provides an indoor air pollution-free cleanroom system achieving artificial intelligence green and healthy building standards, and the indoor air pollution-free cleanroom system includes an indoor air pollution detection and purification system a, a high-efficiency building design structure b, a renewable energy system c, a water resource management system d, and a circular economy and resource recycling e. It allows to achieve low energy consumption and high comfort requirements in green building standards (LEED standards) and healthy building standards (WELL standards), and achieve new artificial intelligence green and healthy building standards, thereby controlling the indoor air pollution to be completely clean, the water pollution to zero, the noise pollution to zero and the net carbon emissions to be zero, maximizing the energy saving, and improving the health and comfort of the living environment.

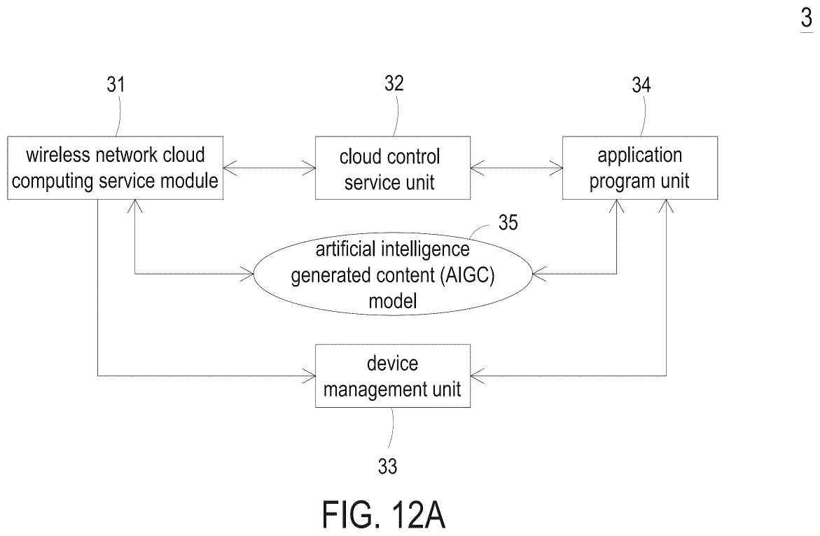

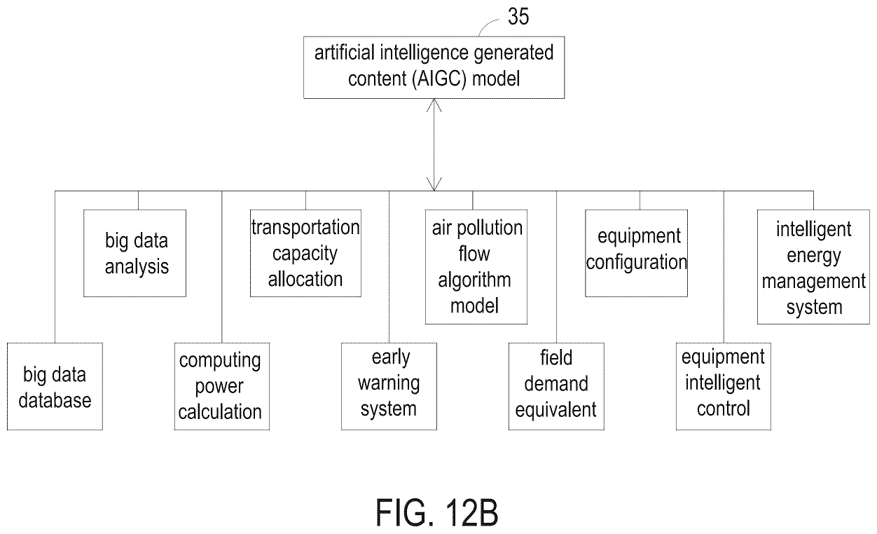

As shown in FIG. 1B and FIG. 1D, in the embodiment, the indoor air pollution detection and purification system a includes a plurality of gas detectors 1, at least one indoor air pollution treatment device 2, at least one networked cloud computing service device 3, at least one storage and data processing device 4 and a central control computer intelligent control device 5. As shown FIG. 1C and FIG. 1D, in the embodiment, the indoor air pollution treatment device 2 includes a gas exchange device 2a, a purifier 2b, a fan filter unit (FFU) 2c, an exhaust device 2d, a cooling and heating device 2e, a range hood 2f, a humidity control device 2g and a mobile vacuum cleaner 2h. Preferably but not exclusively, the gas exchange device 2a provides ventilation for the indoor field A and positive pressure air intake to prevent air pollution from entering the indoor field A. Preferably but not exclusively, the purifier 2b, the fan filter unit (FFU) 2c, the exhaust device 2d, the range hood 2f and the mobile vacuum cleaner 2h provide air pollution purification and clean room treatment for the indoor field A. Preferably but not exclusively, the cooling and heating device 2e and the humidity control device 2g provide temperature and humidity adjustment for the indoor field A. Furthermore, in the embodiment, the indoor air pollution treatment device 2 includes at least one of the plurality of gas detectors 1, at least one air guiding fan 21, at least one filter component 22 and at least one driving controller 23 disposed therein. The included gas detector 1 and the driving controller 23 are electrically connected, and the gas detector 1 receives the control instruction and transmits the control instruction to the driving controller 23 through Internet of Things communication to control actuation operation of the air guiding fan 21. Moreover, the indoor air pollution treatment device 2 integrates HEPA, UVC, activated carbon, photocatalysis, plasma, negative ion and other technologies for intelligent purification. As shown in FIG. 1A and FIG. 12A, the networked cloud computing service device 3 includes a wireless network cloud computing service module 31, a cloud control service unit 32, a device management unit 33, an application unit 34 and an artificial intelligence generated content (AIGC) model 35. In this way, the indoor air pollution detection and purification system a comprehensively monitors indoor air pollution through the plurality of gas detectors 1, and the indoor air pollution treatment device 2 integrates HEPA, UVC, activated carbon, photocatalysis, plasma, negative ion and other technologies for intelligent purification. An intelligent networking system of the networked cloud computing service device 3 and an artificial intelligence generated content (AIGC) model 35 are used to perform dynamic monitoring and adjustment analysis 3, and the operating mode of the indoor air pollution treatment device 2 is adjusted automatically according to real-time data, whereby the indoor air purification effect is achieved, the pollution-free indoor air quality is achieved and the cleanliness of the cleanroom level is achieved.

In the embodiment, the plurality of gas detectors 1 are arranged in an indoor field A and an outdoor field B to detect air pollution and output air quality data through Internet of Things (IoT) communication. The air quality data includes information of suspended particles (PM1, PM2.5, PM10), carbon dioxide (CO₂), volatile organic compounds (VOC), temperature and humidity. The gas detector 1 transmits the data to the networked cloud computing service device 3 via Internet of Things technology, and the artificial intelligence generated content (AIGC) model 35 performs data analysis to generate a corresponding control instruction.

In the embodiment, the indoor air pollution treatment device 2 includes at least one of the gas detectors 1, at least one air guiding fan 21, at least one filter component 22 and at least one driving controller 23 disposed therein. Preferably but not exclusively, the filter component 22 includes HEPA, UVC, activated carbon, photocatalysis, plasma, negative ion and other filtering and purification technologies. In the embodiment, the indoor air pollution treatment device 2 automatically performs the air filtration, the temperature and humidity adjustment, and the sterilization operations according to the control instructions, to keeping the indoor air free of pollutants and achieve the cleanliness of the cleanroom level.

In the embodiment, the indoor air pollution treatment device 2 further includes a central control computer intelligent control device 5, the central control computer intelligent control device 5 receives the control instruction issued by the networked cloud computing service device 3 through Internet of Things communication, and transmits the control instruction received to the gas detector 1 of the indoor air pollution treatment device 2 through Internet of Things communication to control the actuation operation of the air guiding fan 21. In another embodiment, the central control computer intelligent control device 5 includes an edge computing function and allows receiving and analyzing air quality data monitored by each of the gas detectors 1 of the indoor air pollution treatment device 2 through Internet of Things communication, the control instruction is generated based on analysis results and directly issued, and the control instruction is transmitted to a corresponding one of the gas detectors 1 of the indoor air pollution treatment device 2 through Internet of Things communication, and received by the corresponding one of the gas detectors 1, whereby the actuation operation of the air guiding fan 21 is controlled, and automatic control and optimization of the indoor air pollution treatment device 2 is achieved.

In the embodiment, the networked cloud computing service device 3 uses the artificial intelligence generated content (AIGC) model 35 to automatically generate the control instruction and dynamically adjust the operating mode of the indoor air pollution treatment device 2 to achieve energy-saving operation and fault prediction. Notably, the generative artificial intelligence (AIGC) model 35 includes technology of a big data database, a big data analysis, a computing power calculation, a transportation capacity allocation, an early warning system, an air pollution flow algorithm model, a field demand equivalent, an equipment configuration, an equipment intelligent control, an intelligent energy management system, an integration of the system to form AI data calculation global intelligent control, and an integration of the indoor air pollution treatment device 2 to achieve automatic control.

In the embodiment, the generative artificial intelligence (AIGC) model 35 can learn the user habits and environmental changes, and dynamically adjust the equipment operation according to the weather forecasts, the external air quality and the energy consumption requirements to maximize the energy efficiency and optimize the air quality. Preferably but not exclusively, the generative artificial intelligence (AIGC) model 35 includes an intelligent energy management system, and the intelligent energy management system automatically adjusts the operation mode of the indoor air pollution treatment device 2 according to the real-time monitoring data, so as to achieve energy saving effects. That is, the intelligent linkage system formed by the indoor air pollution detection and purification system a can instantly control the actuation operation of the air guiding fan 21, monitor the air quality, the temperature and humidity adjustment in the space of the indoor field A anytime and anywhere. Moreover, the control instruction is issued for driving based on the intelligent comparison of the monitoring status to control the actuation operation of the air guiding fan 21 and the adjustment of the air volume, so as to effectively control the energy-saving benefits of the air-conditioning device. Preferably but not exclusively, the generative artificial intelligence (AIGC) model 35 includes an early warning system to monitor the operation of the equipment and predict the potential failures, and includes a self-cleaning technology, especially automatic cleaning of the filter component 22 and the guide channel 24, which reduces the need for daily maintenance to maintain a long-term and efficient operation of the equipment. Preferably but not exclusively, the generative artificial intelligence (AIGC) model 35 includes an air pollution flow algorithm model to predict the future air quality changes, adjust the equipment operating status in advance, and prevent the sudden changes in air quality. Preferably but not exclusively, the generative artificial intelligence (AIGC) model 35 includes an equipment intelligent control to integrate more types of sensors (such as noise sensors), and intelligently adjust the system operation according to different environmental parameters, thereby improving the operating efficiency and the accuracy of the system.

In the embodiment, the storage and data processing device 4 collects information data from the plurality of gas detectors 1 to store and form a big data database, and the artificial intelligence generated content (AIGC) model 35 captures the big data database through calculation, comparison and recognition, generates the automatic control instruction based on the calculation results and adjusts the operation of the indoor air pollution treatment device 2.

From the above, the indoor air pollution detection and purification system a is disposed in an indoor field A. The networked cloud computing service device 3 receives the air quality data outputted from the plurality of gas detectors 1 through Internet of Things communication, analyzes the air quality data based on the generative artificial intelligence (AIGC) model 35, and intelligently selects to issue the control instruction according to an analyzed result of the air quality data, to automatically adjust an operation mode of the indoor air pollution treatment device 2 for carrying out circulating air pollution purification and completely clean room treatment in the indoor field A, and achieving a cleanliness of cleanroom level.

In the embodiment, the high-efficiency building design structure b includes a passive design structure and an airtight design structure. Preferably but not exclusively, the passive design structure includes a natural ventilation structure, a natural lighting structure and a thermal insulation structure for reducing the need for air conditioning and artificial lighting. The green building materials (such as low-VOC materials) is further combined to reduce the pollutants released by the building materials themselves, and the plant walls and the roof gardens are used to absorb and regulate the carbon dioxide in the air. In the embodiment, the airtight design structure is used to improve a building airtightness and prevent air leakage. At the same time, it is combined with the ventilation of the indoor air pollution detection and purification system a to ensure the air circulation and achieve the purpose of preventing the accumulation of pollutants.

In the embodiment, the renewable energy system c includes a solar photovoltaic system, a wind energy system, a geothermal energy system and an energy storage system (not shown) for providing electricity to a building and storing excess electricity to meet peak electricity demand. Notably, the solar photovoltaic systems can be installed on the roofs and the walls of the buildings to generate clean energy to support the electricity needs of the entire building, especially the operation of air purification equipment. The energy storage system can be installed with an advanced battery energy storage systems to store the excess electricity from the renewable energy and release it during the peak power consumption or at night to ensure the continuous operation of air purification device.

In the embodiment, the water resource management system d includes a rainwater collection system (not shown) and a high-efficiency water-saving device (not shown). The rainwater collection system is used to recycle rainwater. The high-efficiency water-saving device includes a low-flow toilet and an intelligent sprinkler system configured to use rainwater recycled by the rainwater collection system to save water. Notably, the rainwater collection system refers to a system that collects, stores and reuses the rainfall water through the appropriate infrastructure. These systems usually include the rooftop or ground collection surfaces, the collection pipes, the filtration devices and the storage tanks. The purpose of the rainwater collection system is to reduce dependence on the municipal water supply and appropriately use the collected rainwater for non-drinking purposes such as irrigation, flushing toilets, and washing vehicles.

In the embodiment, the circular economy and resource recycling e includes a building material recycling and a construction resource recycling. The building material recycling is configured to recycle building demolition or renovation materials, and the construction resource recycling is configured to promote an efficient use of resources within a building life cycle. Notably, the building material recycling refers to the process of recycling and reusing dismantled building materials during the demolition or renovation of buildings. Preferably but not exclusively, these materials include metals, concrete, bricks, wood, and glass. Through screening, processing and producing, these materials can be used in new construction projects or other projects, thereby reducing the waste emissions and lowering the demand for raw materials, and promoting the sustainable development. The building material recycling is a broader concept that covers the rational use and regeneration of resources throughout the life cycle of the building, including design, construction, operation, maintenance and demolition. These process not only include the reuse of building materials, but also emphasize the efficient use of resources (such as water and energy) and the minimization of waste. The goal of recycling is to maximize the service life of building materials, reduce the impact on the environment, and promote the green transformation of the construction industry.

From the above, in the specific implementation of the present disclosure, the intelligently controlled indoor air pollution detection and purification system a and the advanced indoor and outdoor air pollution treatment device 2 are integrated with the efficient building design structure b, the renewable energy system c, the water resource management system d, the circular economy and resource recycling e, and combined with the net zero carbon emission goal and the efficient air purification technology. It allows to achieve low energy consumption and high comfort requirements in green building standards (LEED standards) and healthy building standards (WELL standards), and achieve new artificial intelligence green and healthy building standards, thereby controlling the indoor air pollution to be completely clean, the water pollution to zero, the noise pollution to zero and the net carbon emissions to be zero, and maximizing the energy saving. It allows to achieve carbon neutrality while keeping indoor air free of pollutants. Consequently, the cleanliness of the cleanroom level is achieved, and the health and comfort of the living environment is improved.

The following is a specific example of the indoor air pollution detection and purification system a used in the indoor field A:

Please refer to FIG. 1D. In the embodiment, a plurality of gas detectors 1 are arranged in an indoor field A and an outdoor field B to detect air pollution and output air quality data through Internet of Things (IoT) communication. Notably, in the embodiment, the air quality data includes information of suspended particles (PM1, PM2.5, PM10), carbon dioxide (CO₂), volatile organic compounds (VOC), temperature and humidity.

In the embodiment, the indoor air pollution treatment device 2 is arranged in the indoor field A, and includes at least one of the gas detectors 1, at least one air guiding fan 21, at least one filter component 22 and at least one driving controller 23 disposed therein. In the embodiment, the gas detector 1 and the driving controller 23 are electrically connected, and the gas detector 1 receives a control instruction through Internet of Things communication to control actuation operation of the air guiding fan 21 through the driving controller 23, for carrying out circulating air pollution purification and completely clean room treatment in the indoor field A. The gas exchange device 2a provides ventilation for the indoor field A and positive pressure air intake to prevent air pollution from entering the indoor field A. The purifier 2b, the fan filter unit (FFU) 2c, the exhaust device 2d, the range hood 2f and the mobile vacuum cleaner 2h provide air pollution purification and clean room treatment for the indoor field A. Preferably but not exclusively, the cooling and heating device 2e and the humidity control device 2g provide temperature and humidity adjustment for the indoor field A.

As shown in FIG. 12A, in the embodiment, the networked cloud computing service device 3 includes a wireless network cloud computing service module 31, a cloud control service unit 32, a device management unit 33, an application unit 34 and an artificial intelligence generated content (AIGC) model 35. Preferably but not exclusively, the embodiment, the wireless network cloud computing service module 31 receives the air quality data of the indoor field A and the outdoor field B, receives communication information from the indoor air pollution treatment device 2 and transmits the control instruction. The wireless network cloud computing service module 31 receives the air quality data of the indoor field A and the outdoor field B and transmits the air quality data received to the cloud control service unit 32 to store and form an air pollution big data database. Moreover, the wireless network cloud computing service module 31 implements intelligent calculation and comparison through the air pollution big data database, and issues the control instruction to the wireless network cloud computing service module 31, and then the control instruction is transmitted through the wireless network cloud computing service module 31 to control the actuation operation of the indoor air pollution treatment device 2. The device management unit 33 receives the communication information of the devices through the wireless network cloud computing service module 31 to manage the user login and device binding. The maintenance and management of the indoor air pollution treatment device 2, the automatic abnormal point detection, the analysis, treatment and improvement, the control and inspection measurement for meeting the cleanliness requirements of the clean room level, the customer demand feedback and the correction mechanism for software and hardware technology improvement, and other management information to the application unit for system control can be provided to the application program unit 34 for system control and management, and the application program unit 34 can also display and inform the air quality data information obtained by the cloud control service unit 32. The user can know the real-time status of air pollution removal through the mobile phone or the communication device. Moreover, the user can control the operation of the indoor air pollution detection and purification system a for achieving artificial intelligence green and healthy building standards through the application program unit 34 of the mobile phone or the communication device. In the embodiment, the artificial intelligence generated content (AIGC) model 35 generates automatically-generated data through calculation, comparison and recognition of the professional generated data and the user generated data. An AI data calculation is formed for global intelligent control, the indoor air pollution treatment device 2 is integrated to achieve the automatic control, and the operation mode of the indoor air pollution treatment device 2 is dynamically adjusted, so as to achieve the energy-saving operation and the fault prediction.

Notably, in the embodiment, the networked cloud computing service device 3 receives the air quality data of the indoor field A and the outdoor field B through Internet of Things communication to form an air pollution big data database, and issues a control instruction to the gas detector 1 of the indoor air pollution treatment device 2 according to the intelligent comparison of the air quality data detected. The gas detector 1 is connected to the driving controller 23 to control the actuation operation of control operation of the air guiding fan 21. The networked cloud computing service device 3 receives the air quality data outputted from the plurality of gas detectors 1 through Internet of Things communication, analyzes the air quality data based on the generative artificial intelligence (AIGC) model 35, and intelligently selects to issue the control instruction according to an analyzed result of the air quality data, to automatically adjust an operation mode of the indoor air pollution treatment device 2 for carrying out circulating air pollution purification and completely clean room treatment in the indoor field A, and achieving a cleanliness of cleanroom level.

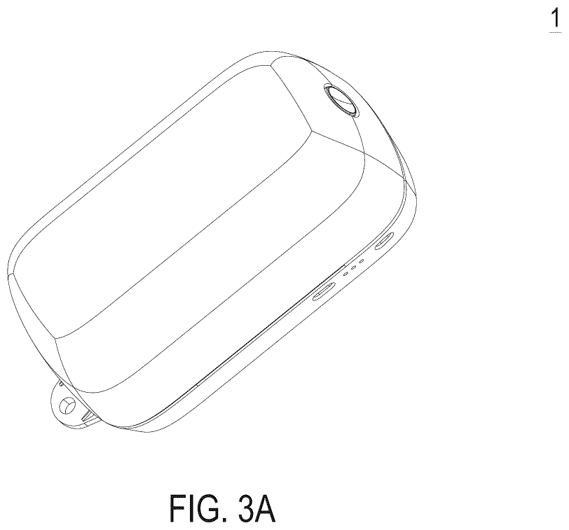

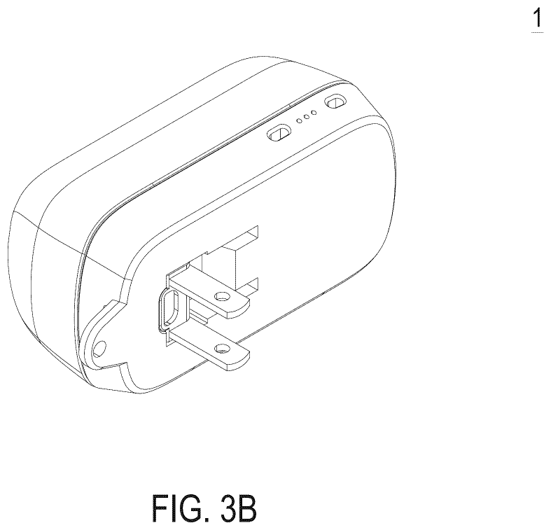

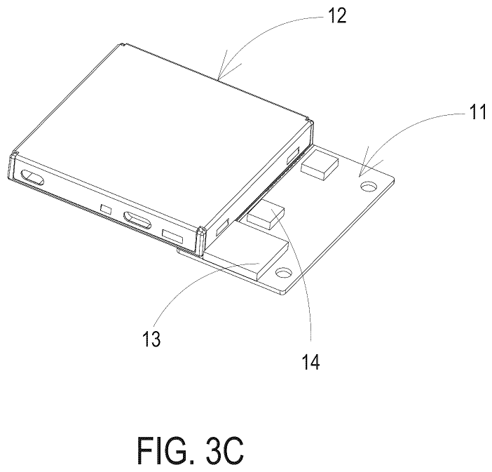

Notably, in the embodiment, the gas detector 1 includes a gas detection module disposed therein. Please refer to FIG. 3A and FIG. 3B. Preferably but not exclusively, the gas detector 1 is formed by a type of structure including an external power terminal, which can be directly inserted into the power interface in the indoor field A to start the operation of detecting the air pollution information, the carbon dioxide (CO₂) pressure detection information, and the gas temperature and humidity information. In an embodiment, as shown in FIG. 3C, a type of the gas detection module without an external power terminal is directly constructed on and electrically connected to an indoor device. A control instruction is received to control the power supply and the actuation operation of the indoor air pollution treatment device 2.

Notably, in the embodiment, the air pollution is at least one selected from the group consisting of particulate matter, carbon monoxide, carbon dioxide, ozone, sulfur dioxide, nitrogen dioxide, lead, total volatile organic compounds (TVOC), formaldehyde, bacteria, fungi, virus and a combination thereof.

In the embodiment, Internet of Things communication refers to a collective network, which connects various devices and technologies and helps the devices communicate with the cloud and with each other. Preferably but not exclusively, Internet of Things communication is a wired communication, which is connected to the networked cloud computing service device 3 via a wired line. Preferably but not exclusively, Internet of Things communication is a wireless communication for communicating with the networked cloud computing service device 3 via a wireless connection. The wireless communication transmission includes one selected from the group consisting of a Wi-Fi module, a Bluetooth module, a radio frequency identification module, and a near field communication (NFC) module.

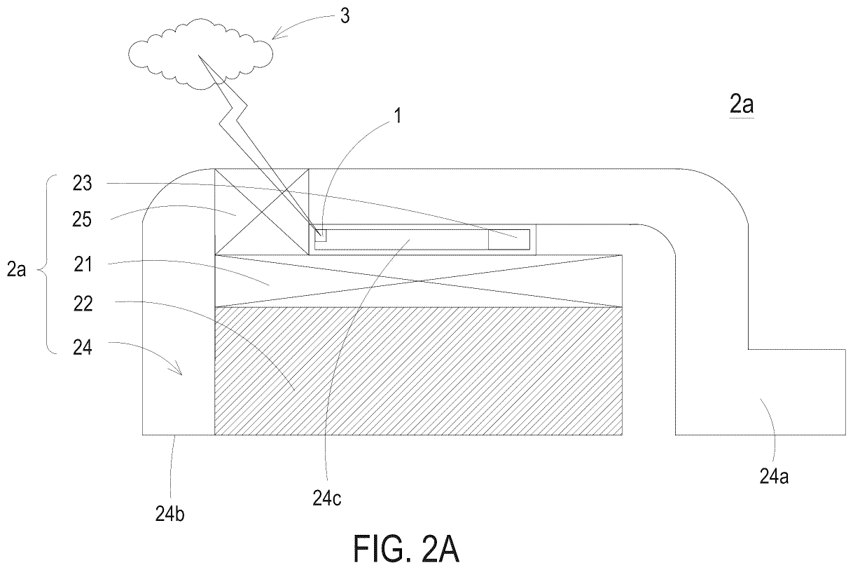

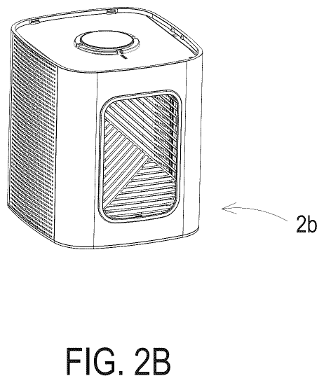

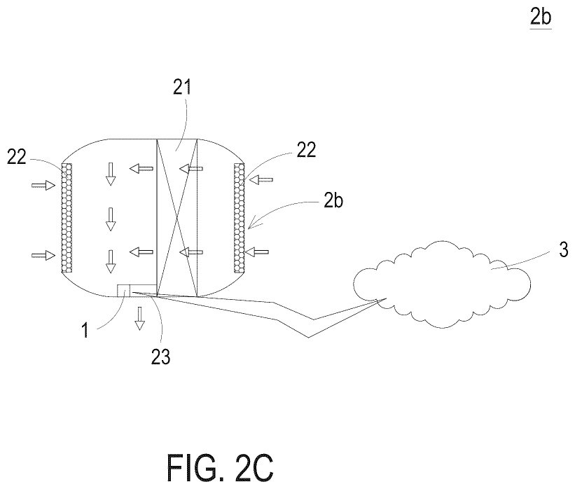

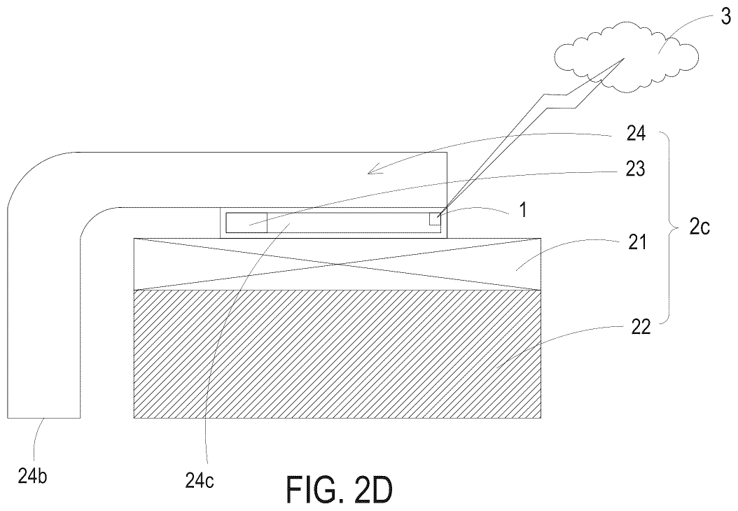

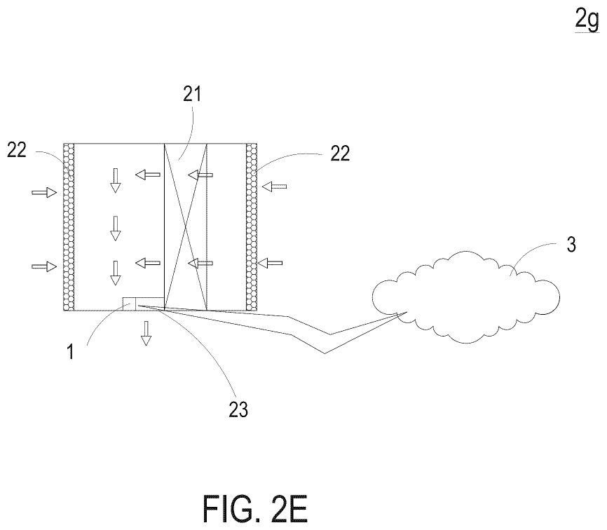

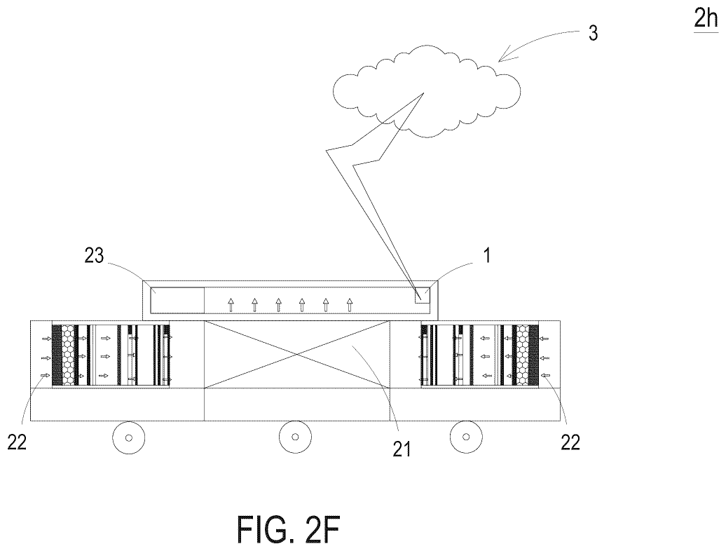

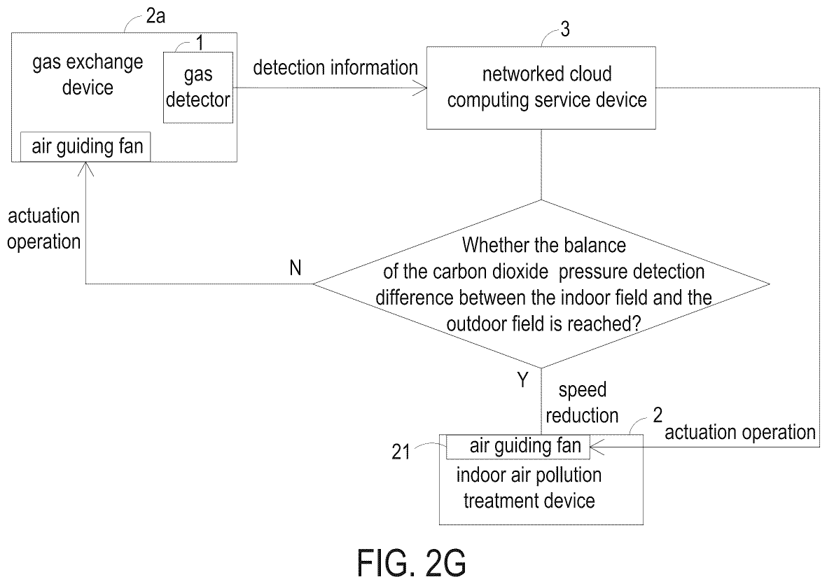

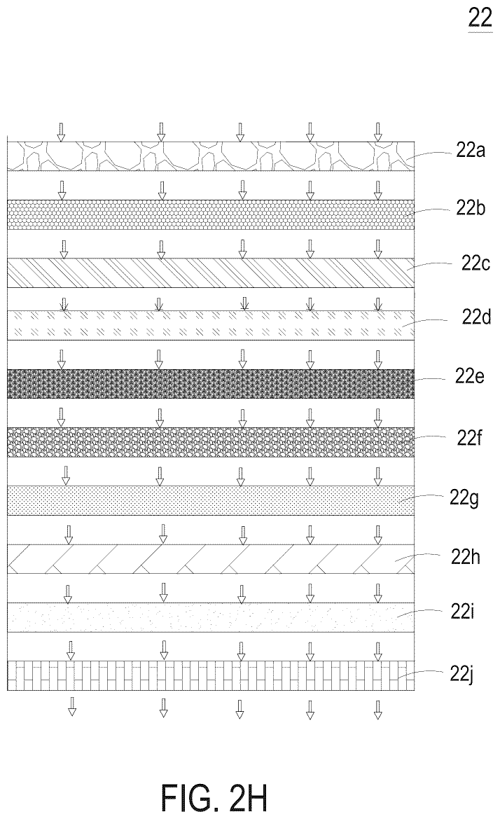

Notably, as shown in FIG. 1D, the indoor air pollution treatment device 2 is arranged in the indoor field A. In the embodiment, the indoor field A includes at least one air inlet C1 and at least one exhaust port C2. As shown in FIG. 1D and FIG. 2A, the gas exchange device 2a includes a guide channel 24. The guide channel 24 has an air inlet 24a corresponding to the air inlet C1 of the indoor field A, a circulating return port 24b in communication with the indoor field A, and a filtering air duct 24c in communication with the indoor field A. A gas exchange fan 25 is disposed in the circulating return port 24b, and an air guiding fan 21 and a filter component 22 are disposed in the filtering air duct 24c. In the embodiment, the networked cloud computing service device 3 intelligently calculates and compares the carbon dioxide (CO₂) pressure detection information of the indoor field A and the outdoor field B. Notably, the safety value of the carbon dioxide (CO₂) pressure detection information in the indoor field A must be maintained at 400 PPM ˜600 PPM. As shown in FIG. 2G, when the networked cloud computing service device 3 receives the detection information of the gas exchange device 2a through the Internet of Things communication, the pressure difference of carbon dioxide (CO₂) in the indoor field A and the outdoor field B are compared to check whether the balance of zero pressure difference is reached. That is to say, the safety values of the carbon dioxide (CO₂) pressure detection information of the indoor field A and the outdoor field B must be the same. When the balance of zero pressure difference is not reached, a control instruction is selected to be issued. The gas detector 1 of the gas exchange device 2a receives and controls the driving controller 23 to control the actuation operation of the air guiding fan 21, so that the air of the outdoor field B is introduced into the filtering air duct 24c through the air inlet C1, and filtered into the indoor field A through the filter component 22. At the same time, the air of the indoor field A enters the filtering air duct 24c through the circulating return port 24b, and then is circulated and filtered to adjust the temperature and implement the ventilation. The ventilation is implemented to achieve the balance of the carbon dioxide (CO₂) pressure detection difference between the indoor field A and the outdoor field B. Notably, when the gas exchange device 2a is enabled for ventilation, a positive pressure greater than 0 Pa is maintained in the space of the indoor field A to prevent the air pollution in the outdoor field B from entering the indoor field A. In an embodiment, the gas detector 1 inside the indoor air pollution treatment device 2 continuously receives the control instruction from the networked cloud computing service device 3 and controls the driving controller 23 to control the actuation operation of the air guiding fan 21, and the internal air pollution of the indoor field A is continuously circulated and purified, so that the internal circulation air pollution purification and complete clean room treatment and the temperature and humidity adjustment of the indoor field A are implemented continuously. When the networked cloud computing service device 3 compares the pressure difference of carbon dioxide (CO₂) between the indoor field A and the outdoor field B to reach the balance of zero pressure difference, the networked cloud computing service device 3 also issues a control instruction to the internal gas detector 1 of the indoor air pollution treatment device 2, and the control instruction is received to control the air guiding fan 21 through the driving controller 23. In that, the air guiding fan 21 is controlled in real time to adjust the guide air volume according to the air quality, the energy-saving efficiency of the operation is effectively adjusted, and the air flow volume noise reaches zero specification value. Consequently, the instant detection of air pollution purification and the complete clean room treatment are realized, and the cleanliness of cleanroom grade is achieved. Notably, as shown in FIG. 1C, the gas exchange device 2a is a fresh air fan. Preferably but not exclusively, the gas exchange device 2a is a full heat exchanger. Preferably but not exclusively, the gas exchange device 2a is a heating ventilation and air conditioning (HVAC).

Please refer to FIG. 1D, FIG. 2B and FIG. 2C. In the embodiment, the purifier 2b is plugged-in to the space of the indoor field A, and the networked cloud computing service device 3 issues the control instruction to the gas detector 1 inside the purifier 2b through the Internet of Things communication. The control instruction is received by the driving controller 23 to control the actuation operation of the air guiding fan 21, so as to guide the air pollution in the space of the indoor field A to be filtered and purified by the filter component 22, and the air purified is then introduced into the space of the indoor field A. Whereby, the air pollution in the space of the indoor field A is guided to pass through the filter component 22 multiple times for air pollution purification and complete clean room treatment.

Please refer to FIG. 1D and FIG. 2D. In the embodiment, the fan filter unit (FFU) 2c is built-in to the space of the indoor field A. The fan filter unit (FFU) 2c includes a guide channel 24. The guide channel 24 has a circulating return port 24b in communication with the indoor field A, and a filtering air duct 24c in communication with the indoor field A. An air guiding fan 21 and a filter component 22 are disposed in the filtering air duct 24c. The networked cloud computing service device 3 issues the control instruction to the gas detector 1 inside the fan filter unit (FFU) 2c through the Internet of Things communication. The control instruction is received by the gas detector 1 and transmitted to the driving controller 23 to control the actuation operation of the air guiding fan 21. In that, the air pollution in the space of the indoor field A is guided from the circulating return port 24b into the filtering air duct 24c of the guide channel 24 to be filtered and purified by the filter component 22 inside the fan filter unit (FFU) 2c. The air purified is then introduced into the space of the indoor field A from the guide channel 24, so that the air pollution in the space of the indoor field A is guided to enter the guide channel 24 multiple times for air pollution purification and complete clean room treatment.

Please refer to FIG. 1D. In the embodiment, the exhaust device 2d is built-in to the indoor field A, corresponds to the exhaust port C2 and is in communication with the outdoor field B. The networked cloud computing service device 3 issues the control instruction to the gas detector 1 inside the exhaust device 2d through the Internet of Things communication. The control instruction is received by the driving controller 23 to control the actuation operation of the air guiding fan 21 to guide the air pollution in the space of the indoor field A to be introduced by the air guiding fan 21 through the filter component 22 for filtration and purification and discharged to the outdoor field B. Whereby, the air pollution in the space of the indoor field A tis purified to achieve the complete clean room treatment.

Please refer to FIG. 1D. In the embodiment, the cooling and heating device 2e is disposed in the indoor field A. The cooling and heating device 2e includes a temperature regulating exchanger 26. The networked cloud computing service device 3 issues the control instruction to the gas detector 1 inside the cooling and heating device 2e through the Internet of Things communication. The control instruction is received by the gas detector 1 and transmitted to the driving controller 23 to control the actuation operation of the air guiding fan 21. The air is introduced to pass through the temperature regulating exchanger 26 to regulate the temperature and humidity of the air in the indoor field A, and the gas detector 1 externally transmits the temperature and humidity information of the air in the indoor field A. Notably, in the embodiment, the gas temperature and humidity in the indoor field A are adjusted by the cooling and heating device 2e to maintain the temperature at 25° C.±3° C. and the humidity at 50%±10%. Notably, as shown in FIG. 1C, the cooling and heating device 2e is a cooling exchanger, or the cooling and heating device 2e is a heating exchanger. The present disclosure is not limited thereto.

Please refer to FIG. 1D. When cooking food in the kitchen environment of indoor field A, serious air pollution is generated relatively quickly. In order to prevent the air pollution generated in indoor field A from affecting human health and causing harm, the indoor air pollution treatment device 2 of the indoor air pollution detection and purification system a for achieving artificial intelligence green and healthy building standards further includes a range hood 2f, which is set and arranged in the kitchen of indoor field A. The range hood 2f includes an exhaust channel 2fa, which corresponds to the exhaust port C2 in communicate with the outdoor field B, and is arranged above a cooking appliance H. The exhaust channel 2fa is provided with an air guiding fan 21, a filter component 22 and a driving controller 23, and the gas detector 1 is arranged in the exhaust channel 2fa and electrically connected to the driving controller 23. The gas detector 1 receives the control instruction to the driving controller 23 through the Internet of Things communication to control the actuation operation of the air guiding fan 21. Moreover, the range hood 2f includes an oil fume exhaust body 2fb, which corresponds to the exhaust port C2 in communication with the outdoor field B and is arranged in front of the cooking appliance H. The oil fume exhaust body 2fb is provided with an air guiding fan 21, a filter component 22 and a driving controller 23, and a gas detector 1 is arranged in the oil fume exhaust body 2fb and electrically connected to the driving controller 23. The control instruction is received by the gas detector 1 to the driving controller 23 through the Internet of Things communication to control the actuation operation of the air guiding fan 21. The networked cloud computing service device 3 issues the control instruction to the gas detector 1 inside the exhaust channel 2fa and the gas detector 1 inside the oil fume exhaust body 2fb of the range hood 2f through the Internet of Things communication. The control instruction is transmitted to the driving controller 23 to control the actuation operation of the air guiding fan 21. The air pollution in the kitchen of the indoor field A flows into the exhaust channel 2fa and the oil fume exhaust body 2fb and passes through the filter component 22 therein for filtering and purification, and then introduced into the outdoor field B, so that the kitchen for air pollution purification and complete clean room treatment is achieved.

Please refer to FIG. 1D and FIG. 2E. In the embodiment, the humidity control device 2g is plugged-in to the space of the indoor field A. The networked cloud computing service device 3 issues the control instruction to the gas detector 1 inside the humidity control device 2g through the Internet of Things communication. The control instruction is received by the gas detector 1 and transmitted to the driving controller 23 to control the actuation operation of the air guiding fan 21. The air pollution in the space of the indoor field A is guided to the filter component 22 for air pollution purification and complete clean room treatment, and the gas temperature and humidity in the indoor field A are adjusted. Notably, in the embodiment, the gas temperature and humidity in the indoor field A are adjusted by the humidity control device 2g to maintain the temperature at 25° C.±3° C. and the humidity at 50%±10%. Notably, as shown in FIG. 1C, the humidity control device 2g is a dehumidifier, or the humidity control device 2g is a humidifier. In an embodiment, the humidity control device 2g includes a dehumidifier and a humidifier. The present disclosure is not limited thereto.

Pease refer to FIG. 1D and FIG. 2F. In the embodiment, the mobile vacuum cleaner 2h is plugged-in to the space of the indoor field A, and the networked cloud computing service device 3 issues the control instruction to the gas detector 1 inside the mobile vacuum cleaner 2h through the Internet of Things communication. The control instruction is received by the driving controller 23 to control the actuation operation of the air guiding fan 21. The air pollution in the space of the indoor field A is guided to the filter component 22 for air pollution purification and complete clean room treatment. Notably, the mobile vacuum cleaner 2h is a sweeping robot.

Notably, as shown in FIG. 1A, in the embodiment, the storage and data processing device 4 collects information data of the system to form a big data database of professional generated data and user generated data, and generates automatically-generated data through calculation, comparison and recognition of the generative artificial intelligence (AIGC) model 35. In that, the control instruction is automatically generated and the operating mode is dynamically adjusted to achieve energy-saving operation and fault prediction.

As shown in FIG. 12B, in the embodiment, the generative artificial intelligence (AIGC) model 35 includes an intelligent energy management system, and the intelligent energy management system automatically adjusts the operation mode of the equipment according to the real-time monitoring data, so as to achieve energy saving effects. That is, the intelligent linkage system formed by the indoor air pollution detection and purification system a can instantly control the actuation operation of the air guiding fan 21, monitor the air quality, the temperature and humidity adjustment in the space of the indoor field A anytime and anywhere. Moreover, the control instruction is issued for driving based on the intelligent comparison of the monitoring status to control the actuation operation of the air guiding fan 21 and the adjustment of the air volume, so as to effectively control the energy-saving benefits of the air-conditioning device. In the embodiment, the generative artificial intelligence (AIGC) model 35 includes a system maintenance diagnosis to monitor the operation of the equipment and predict the potential failures, and includes a self-cleaning technology, especially automatic cleaning of the filter component 22 and the guide channel 24, which reduces the need for daily maintenance to maintain a long-term and efficient operation of the equipment. In the embodiment, the generative artificial intelligence (AIGC) model 35 includes an air quality prediction model to predict the future air quality changes, adjust the equipment operating status in advance, and prevent the sudden changes in air quality. In the embodiment, the generative artificial intelligence (AIGC) model 35 includes an intelligent environmental sensing system to integrate more types of sensors (such as noise sensors), and intelligently adjust the system operation according to different environmental parameters, thereby improving the operating efficiency and the accuracy of the system.

From the above, the present disclosure provides an indoor air pollution-free cleanroom system achieving artificial intelligence green and healthy building standards. The net zero carbon emission goal is combined with the high-efficiency air purification technology, and it allows to achieve carbon neutrality while keeping indoor air free of pollutants. The intelligently controlled indoor air pollution detection and purification system a is integrated with the efficient building design structure b, the renewable energy system c, the water resource management system d, the circular economy and resource recycling e, and the advanced indoor and outdoor air pollution treatment device 2. It allows to achieve the efficient energy utilization and the comprehensive air purification, and keep the indoor air pollutants close to free, so that the cleanliness of cleanroom level is achieved.

Notably, the required cleanliness of the indoor field A of the present disclosure needs to meet a clean room level of ZAPClean Room 1 to ZAPClean Room 12. The following is an explanation of the level calibration of ZAPClean room 1 to ZAPClean Room 12:

The air pollution data for the gas state of the air pollution in a required indoor field space of the indoor field A is targeted according to a cumulative number of 21,000 inhaled suspended particles ranged from 1 nm to 2.5 um and detected in 24 hours, and includes detection of suspended particulate matter PM2.5≤0.000000012 μg/m³, detection of suspended particulate matter PM10≤0.00000019 μ82 g/m³, detection of bacteria≤0 CFU/m³, detection of fungi≤0 CFU/m³, detection of formaldehyde≤0.00028 ppm/hour, detection of volatile organic compounds (TVOC)≤0.00094 ppm/hour, detection of carbon dioxide≤500˜650 ppm per 8 hours, detection of carbon monoxide≤0.03149 ppm per 8 hours, and detection of ozone≤0.00021 ppm per 8 hours, for meeting the clean room requirement of ZAPClean Room 1.

The air pollution data for the gas state of the air pollution in a required indoor field space of the indoor field A is targeted according to a cumulative number of 210,000 inhaled suspended particles ranged from 1 nm to 2.5 um and detected in 24 hours, and includes detection of suspended particulate matter PM2.5≤0.00000012 μg/m³, detection of suspended particulate matter PM10≤0.0000019 μg/m³, detection of bacteria≤0 CFU/m³, detection of fungi≤0 CFU/m³, detection of formaldehyde≤0.00047 ppm/hour, detection of volatile organic compounds (TVOC)≤0.00157 ppm/hour, detection of carbon dioxide≤500˜650 ppm per 8 hours, detection of carbon monoxide≤0.05249 ppm per 8 hours, and detection of ozone≤0.00035 ppm per 8 hours, for meeting the clean room requirement of ZAPClean Room 2.

The air pollution data for the gas state of the air pollution in a required indoor field space of the indoor field A is targeted according to a cumulative number of 2,100,000 inhaled suspended particles ranged from 1 nm to 2.5 μm and detected in 24 hours, and includes detection of suspended particulate matter PM2.5≤0.00000124 μg/m³, detection of suspended particulate matter PM10≤0.000019 μg/m³, detection of bacteria≤0 CFU/m³, detection of fungi≤0 CFU/m³, detection of formaldehyde≤0.00078 ppm/hour, detection of volatile organic compounds (TVOC)≤0.00261 ppm/hour, detection of carbon dioxide≤500˜650 ppm per 8 hours, detection of carbon monoxide≤0.08748 ppm per 8 hours, and detection of ozone≤0.00058 ppm per 8 hours, for meeting the clean room requirement of ZAPClean Room 3.

The air pollution data for the gas state of the air pollution in a required indoor field space of the indoor field A is targeted according to a cumulative number of 21,000,000 inhaled suspended particles ranged from 1 nm to 2.5 um and detected in 24 hours, and includes detection of suspended particulate matter PM2.5≤0.00001235 μg/m³, detection of suspended particulate matter PM10≤0.000185 μg/m³, detection of bacteria≤0 CFU/m³, detection of fungi≤0 CFU/m³, detection of formaldehyde≤0.00130 ppm/hour, detection of volatile organic compounds (TVOC)≤0.00435 ppm/hour, detection of carbon dioxide≤500˜650 ppm per 8 hours, detection of carbon monoxide≤0.14580 ppm per 8 hours, and detection of ozone≤0.00097 ppm per 8 hours, for meeting the clean room requirement of ZAPClean Room 4.

The air pollution data for the gas state of the air pollution in a required indoor field space of the indoor field A is targeted according to a cumulative number of 210,000,000 inhaled suspended particles ranged from 1 nm to 2.5 um and detected in 24 hours, and includes detection of suspended particulate matter PM2.5≤0.00123529 μg/m³, detection of suspended particulate matter PM10≤0.01853 μg/m³, detection of bacteria≤1 CFU/m³, detection of fungi≤1 CFU/m³, detection of formaldehyde≤0.00216 ppm/hour, detection of volatile organic compounds (TVOC)≤0.00726 ppm/hour, detection of carbon dioxide≤500˜650 ppm per 8 hours, detection of carbon monoxide≤0.24300 ppm per 8 hours, and detection of ozone≤0.00162 ppm per 8 hours, for meeting the clean room requirement of ZAPClean Room 5.

The air pollution data for the gas state of the air pollution in a required indoor field space of the indoor field A is targeted according to a cumulative number of 2,100,000,000 inhaled suspended particles ranged from 1 nm to 2.5 um and detected in 24 hours, and includes detection of suspended particulate matter PM2.5≤0.00123529 μg/m³, detection of suspended particulate matter PM10≤0.0185294 μg/m³, detection of bacteria≤3 CFU/m³, detection of fungi≤3 CFU/m³, detection of formaldehyde≤0.00360 ppm/hour, detection of volatile organic compounds (TVOC)≤0.01210 ppm/hour, detection of carbon dioxide≤500˜650 ppm per 8 hours, detection of carbon monoxide≤0.40500 ppm per 8 hours, and detection of ozone≤0.00270 ppm per 8 hours, for meeting the clean room requirement of ZAPClean Room 6.

The air pollution data for the gas state of the air pollution in a required indoor field space of the indoor field A is targeted according to a cumulative number of 21,000,000,000 inhaled suspended particles ranged from 1 nm to 2.5 um and detected in 24 hours, and includes detection of suspended particulate matter PM2.5≤0.01235294 μg/m³, detection of suspended particulate matter PM10≤0.0185294 μg/m³, detection of bacteria≤8 CFU/m³, detection of fungi≤8 CFU/m³, detection of formaldehyde≤0.00600 ppm/hour, detection of volatile organic compounds (TVOC)≤0.02016 ppm/hour, detection of carbon dioxide≤500˜650 ppm per 8 hours, detection of carbon monoxide≤0.67500 ppm per 8 hours, and detection of ozone≤0.00450 ppm per 8 hours, for meeting the clean room requirement of ZAPClean Room 7.

The air pollution data for the gas state of the air pollution in a required indoor field space of the indoor field A is targeted according to a cumulative number of 105,000,000,000 inhaled suspended particles ranged from 1 nm to 2.5 um and detected in 24 hours, and includes detection of suspended particulate matter PM2.5≤0.06176471 μg/m³, detection of suspended particulate matter PM10≤0.0926471 μg/m³, detection of bacteria≤15 CFU/m³, detection of fungi≤15 CFU/m³, detection of formaldehyde≤0.009 ppm/hour, detection of volatile organic compounds (TVOC)≤0.02688 ppm/hour, detection of carbon dioxide≤500˜800 ppm per 8 hours, detection of carbon monoxide≤1.0125 ppm per 8 hours, and detection of ozone≤0.00675 ppm per 8 hours, for meeting the clean room requirement of ZAPClean Room 8.

The air pollution data for the gas state of the air pollution in a required indoor field space of the indoor field A is targeted according to a cumulative number of 210,000,000,000 inhaled suspended particles ranged from 1 nm to 2.5 um and detected in 24 hours, and includes detection of suspended particulate matter PM2.5≤0.12 μg/m³, detection of suspended particulate matter PM10≤0.1852941 μg/m³, detection of bacteria≤20 CFU/m³, detection of fungi≤20 CFU/m³, detection of formaldehyde≤0.012 ppm/hour, detection of volatile organic compounds (TVOC)≤0.0336 ppm/hour, detection of carbon dioxide≤500˜800 ppm per 8 hours, detection of carbon monoxide≤1.35 ppm per 8 hours, and detection of ozone≤0.009 ppm per 8 hours, for meeting the clean room requirement of ZAPClean Room 9.

The air pollution data for the gas state of the air pollution in a required indoor field space of the indoor field A is targeted according to a cumulative number of 1,050,000,000,000 inhaled suspended particles ranged from 1 nm to 2.5 um and detected in 24 hours, and includes detection of suspended particulate matter PM2.5≤0.62 μg/m³, detection of suspended particulate matter PM10≤0.9264706 μg/m³, detection of bacteria≤100 CFU/m³, detection of fungi≤80 CFU/m³, detection of formaldehyde≤0.018 ppm/hour, detection of volatile organic compounds (TVOC)≤0.0728 ppm/hour, detection of carbon dioxide≤500˜800 ppm per 8 hours, detection of carbon monoxide≤2.025 ppm per 8 hours, and detection of ozone≤0.0135 ppm per 8 hours, for meeting the clean room requirement of ZAPClean Room 10.

The air pollution data for the gas state of the air pollution in a required indoor field space of the indoor field A is targeted according to a cumulative number of 2,100,000,000,000 inhaled suspended particles ranged from 1 nm to 2.5 um and detected in 24 hours, and includes detection of suspended particulate matter PM2.5≤1.24 μg/m³, detection of suspended particulate matter PM10≤1.85 μg/m³, detection of bacteria≤200 CFU/m³, detection of fungi≤150 CFU/m³, detection of formaldehyde≤0.024 ppm/hour, detection of volatile organic compounds (TVOC)≤0.112 ppm/hour, detection of carbon dioxide≤500˜800 ppm per 8 hours, detection of carbon monoxide≤2.7 ppm per 8 hours, and detection of ozone≤0.018 ppm per 8 hours, for meeting the clean room requirement of ZAPClean Room 11.

The air pollution data for the gas state of the air pollution in a required indoor field space of the indoor field A is targeted according to a cumulative number of 21,000,000,000,000 inhaled suspended particles ranged from 1 nm to 2.5 um and detected in 24 hours, and includes detection of suspended particulate matter PM2.5≤12.35 μg/m³, detection of suspended particulate matter PM10≤18.53 μg/m³, detection of bacteria≤1500 CFU/m³, detection of fungi≤750 CFU/m³, detection of formaldehyde≤0.08 ppm/hour, detection of volatile organic compounds (TVOC)≤0.156 ppm/hour, detection of carbon dioxide≤500˜1000 ppm per 8 hours, detection of carbon monoxide≤9 ppm per 8 hours, and detection of ozone≤0.06 ppm per 8 hours, for meeting the clean room requirement of ZAPClean Room 12.

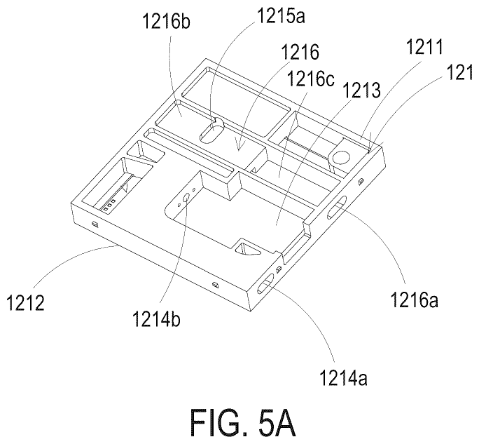

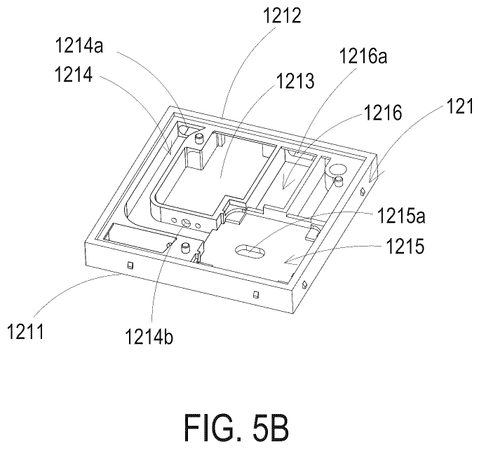

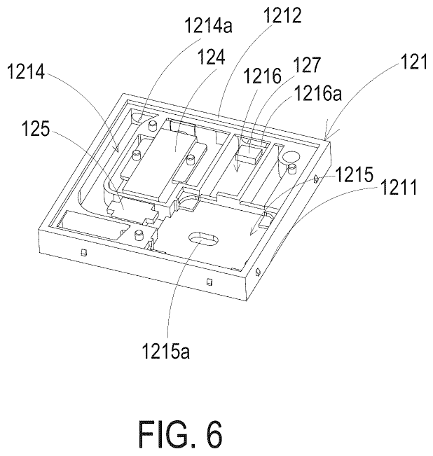

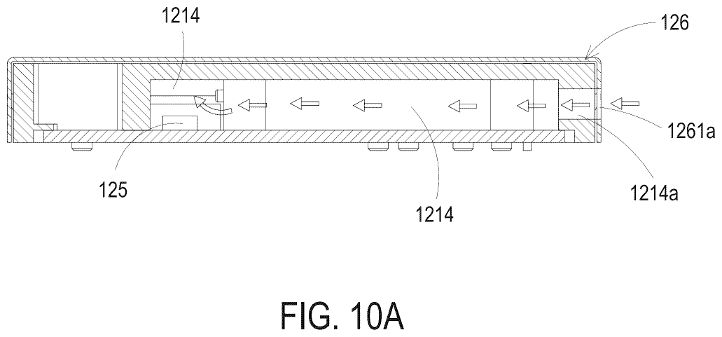

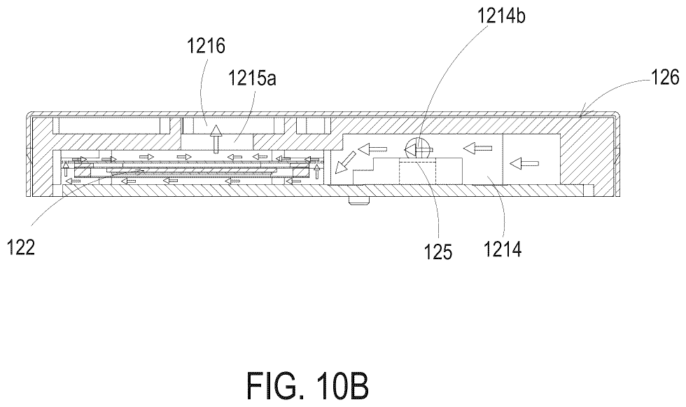

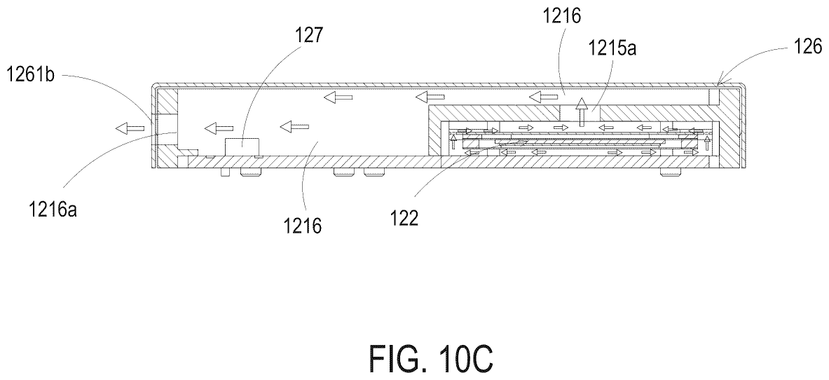

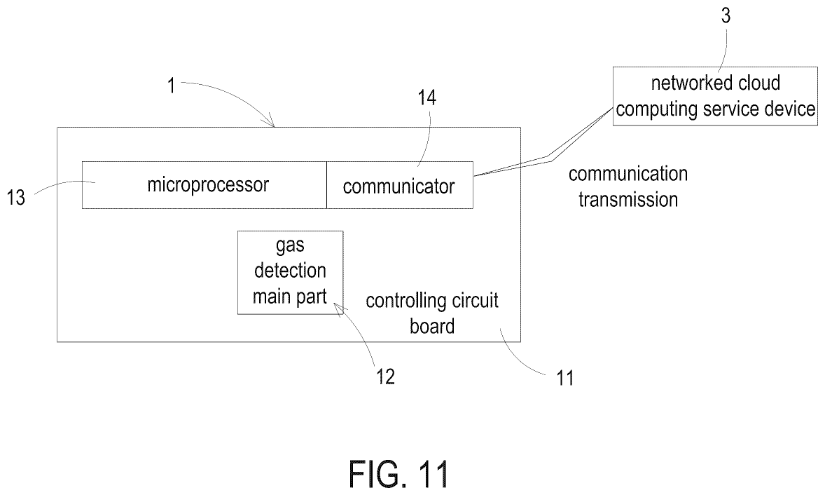

In the present disclosure, the specific implementation of the indoor air pollution-free cleanroom system for achieving artificial intelligence green and healthy building standards is understandable, and the structure of the gas detection module of the gas detector 1 of the present disclosure is described in detail below. Please refer to FIG. 3A to FIG. 11. In the embodiment, the gas detection module includes a controlling circuit board 11, a gas detection main part 12, a microprocessor 13 and a communicator 14. The gas detection main part 12, the microprocessor 13 and the communicator 14 are integrally packaged on the controlling circuit board 11 and electrically connected to each other. In the embodiment, the microprocessor 13 and the communicator 14 are mounted on the controlling circuit board 11. The microprocessor 13 controls the driving signal of the gas detection main part 12 for enabling the detection. In this way, the gas detection main part 12 detects the air pollution and outputs the detection information, and the microprocessor 13 receives, processes and provides the detection information to the communicator 14 for a communication transmission externally, and transmitting to the cloud computing server device 3 through IoT (Internet of Things) communication.

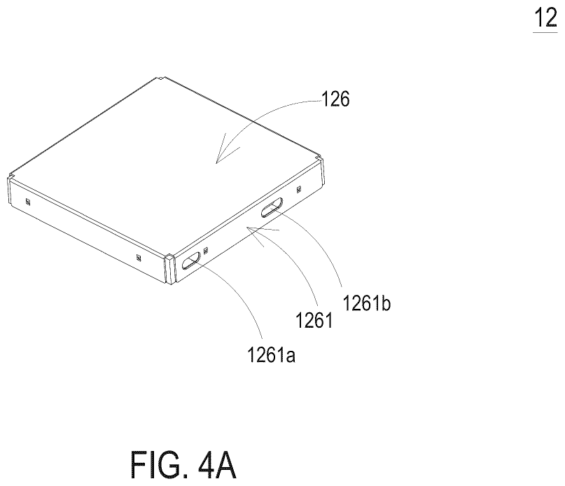

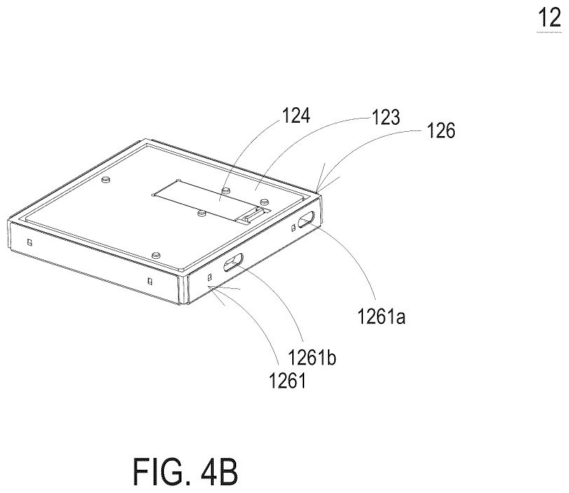

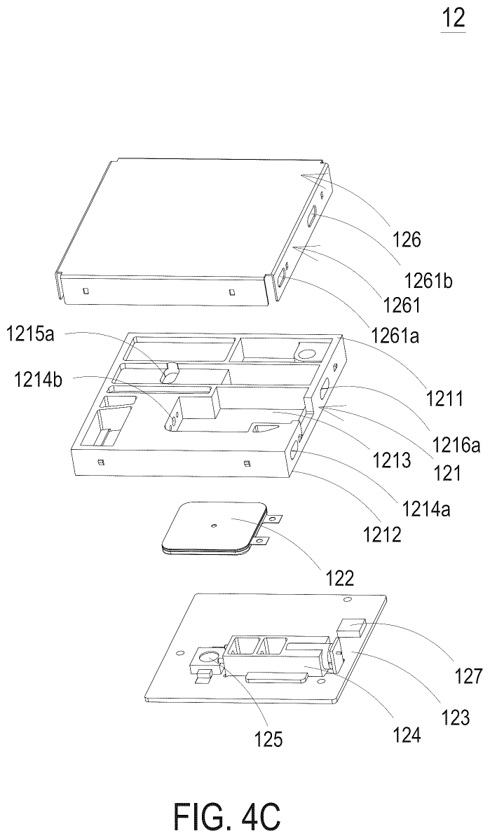

Please refer to FIG. 4A to FIG. 9A. In the embodiment, the gas detection main part 12 includes a base 121, a piezoelectric actuator 122, a driving circuit board 123, a laser component 124, a particulate sensor 125, and an outer cover 126. In the embodiment, the base 121 includes a first surface 1211, a second surface 1212, a laser loading region 1213, a gas-inlet groove 1214, a gas-guiding-component loading region 1215 and a gas-outlet groove 1216. The first surface 1211 and the second surface 1212 are two surfaces opposite to each other. In the embodiment, the laser loading region 1213 is hollowed out from the first surface 1211 toward the second surface 1212. The outer cover 126 covers the base 121 and includes a side plate 1261. The side plate 1261 has an inlet opening 1261a and an outlet opening 1261b. The gas-inlet groove 1214 is concavely formed from the second surface 1212 and disposed adjacent to the laser loading region 1213. The gas-inlet groove 1214 includes a gas-inlet 1214a and two lateral walls. The gas-inlet 1214a is in communication with an environment outside the base 121, and is spatially corresponding in position to an inlet opening 1261a of the outer cover 126. Two transparent windows 1214b are opened on the two lateral walls of the gas-inlet groove 1214 and are in communication with the laser loading region 1213. Therefore, the first surface 1211 of the base 121 is covered and attached by the outer cover 126, and the second surface 1212 is covered and attached by the driving circuit board 123, so that an inlet path is defined by the gas-inlet groove 1214. In the embodiment, the gas-guiding-component loading region 1215 mentioned above is concavely formed from the second surface 1212 and in communication with the gas-inlet groove 1214. A ventilation hole 1215a penetrates a bottom surface of the gas-guiding-component loading region 1215. The gas-guiding-component loading region 1215 includes four positioning protrusions 1215b disposed at four corners of the gas-guiding-component loading region 1215, respectively. In the embodiment, the gas-outlet groove 1216 includes a gas-outlet 1216a, and the gas-outlet 1216a is spatially corresponding to the outlet opening 1261b of the outer cover 126. The gas-outlet groove 1216 includes a first section 1216b and a second section 1216c. The first section 1216b is concavely formed out from the first surface 1211 in a region spatially corresponding to a vertical projection area of the gas-guiding-component loading region 1215. The second section 1216c is hollowed out from the first surface 1211 to the second surface 1212 in a region where the first surface 1211 is extended from the vertical projection area of the gas-guiding-component loading region 1215. The first section 1216b and the second section 1216c are connected to form a stepped structure. Moreover, the first section 1216b of the gas-outlet groove 1216 is in communication with the ventilation hole 1215a of the gas-guiding-component loading region 1215, and the second section 1216c of the gas-outlet groove 1216 is in communication with the gas-outlet 1216a. In that, when first surface 1211 of the base 121 is attached and covered by the outer cover 126 and the second surface 1212 of the base 121 is attached and covered by the driving circuit board 123, the gas-outlet groove 1216 and the driving circuit board 123 collaboratively define an outlet path.

In the embodiment, the laser component 124 and the particulate sensor 125 are disposed on and electrically connected to the driving circuit board 123 and located within the base 121. In order to clearly describe and illustrate the positions of the laser component 124 and the particulate sensor 125 in the base 121, the driving circuit board 123 is intentionally omitted. The laser component 124 is accommodated in the laser loading region 1213 of the base 121, and the particulate sensor 125 is accommodated in the gas-inlet groove 1214 of the base 121 and is aligned to the laser component 124. In addition, the laser component 124 is spatially corresponding to the transparent window 1214b, therefore, a light beam emitted by the laser component 124 passes through the transparent window 1214b and is irradiated into the gas-inlet groove 1214. A light beam path emitted from the laser component 124 passes through the transparent window 1214b and extends in an orthogonal direction perpendicular to the gas-inlet groove 1214. In the embodiment, a projecting light beam emitted from the laser component 124 passes through the transparent window 1214b and enters the gas-inlet groove 1214 to irradiate the suspended particles contained in the gas passing through the gas-inlet groove 1214. When the suspended particles contained in the gas are irradiated and generate scattered light spots, the scattered light spots are received and calculated by the particulate sensor 125 to obtain the gas detection information.

In the embodiment, the piezoelectric actuator 122 is accommodated in the square-shaped gas-guiding-component loading region 1215 of the base 121. In addition, the gas-guiding-component loading region 1215 of the base 121 is in fluid communication with the gas-inlet groove 1214. When the piezoelectric actuator 122 is enabled, the gas in the gas-inlet groove 1214 is inhaled by the piezoelectric actuator 122, so that the gas flows into the piezoelectric actuator 122, and is transported into the gas-outlet groove 1216 through the ventilation hole 1215a of the gas-guiding-component loading region 1215. Moreover, the driving circuit board 123 covers the second surface 1212 of the base 121, and the laser component 124 is disposed on the driving circuit board 123, and is electrically connected to the driving circuit board 123. The particulate sensor 125 is also disposed on the driving circuit board 123 and electrically connected to the driving circuit board 123. In that, when the outer cover 126 covers the base 121, the inlet opening 1261a is spatially corresponding to the gas-inlet 1214a of the base 121, and the outlet opening 1261b is spatially corresponding to the gas-outlet 1216a of the base 121.

In the embodiment, the piezoelectric actuator 122 includes a gas-injection plate 1221, a chamber frame 1222, an actuator element 1223, an insulation frame 1224 and a conductive frame 1225. In the embodiment, the gas-injection plate 1221 is made by a flexible material and includes a suspension plate 1221a and a hollow aperture 1221b. The suspension plate 1221a is a sheet structure and is permitted to undergo a bending deformation. Preferably but not exclusively, the shape and the size of the suspension plate 1221a are accommodated in the inner edge of the gas-guiding-component loading region 1215, but not limited thereto. The hollow aperture 1221b passes through a center of the suspension plate 1221a, so as to allow the gas to flow therethrough. Preferably but not exclusively, in the embodiment, the shape of the suspension plate 1221a is selected from the group consisting of a square, a circle, an ellipse, a triangle and a polygon, but not limited thereto.

In the embodiment, the chamber frame 1222 is carried and stacked on the gas-injection plate 1221. In addition, the shape of the chamber frame 1222 is corresponding to the gas-injection plate 1221. The actuator element 1223 is carried and stacked on the chamber frame 1222. A resonance chamber 1226 is collaboratively defined by the actuator element 1223, the chamber frame 1222 and the suspension plate 1221a and is formed between the actuator element 1223, the chamber frame 1222 and the suspension plate 1221a. The insulation frame 1224 is carried and stacked on the actuator element 1223 and the appearance of the insulation frame 1224 is similar to that of the chamber frame 1222. The conductive frame 1225 is carried and stacked on the insulation frame 1224, and the appearance of the conductive frame 1225 is similar to that of the insulation frame 1224. In addition, the conductive frame 1225 includes a conducting pin 1225a and a conducting electrode 1225b. The conducting pin 1225a is extended outwardly from an outer edge of the conductive frame 1225, and the conducting electrode 1225b is extended inwardly from an inner edge of the conductive frame 1225. Moreover, the actuator element 1223 further includes a piezoelectric carrying plate 1223a, an adjusting resonance plate 1223b and a piezoelectric plate 1223c. The piezoelectric carrying plate 1223a is carried and stacked on the chamber frame 1222. The adjusting resonance plate 1223b is carried and stacked on the piezoelectric carrying plate 1223a. The piezoelectric plate 1223c is carried and stacked on the adjusting resonance plate 1223b. The adjusting resonance plate 1223b and the piezoelectric plate 1223c are accommodated in the insulation frame 1224. The conducting electrode 1225b of the conductive frame 1225 is electrically connected to the piezoelectric plate 1223c. In the embodiment, the piezoelectric carrying plate 1223a and the adjusting resonance plate 1223b are made by a conductive material. The piezoelectric carrying plate 1223a includes a piezoelectric pin 1223d. The piezoelectric pin 1223d and the conducting pin 1225a are electrically connected to a driving circuit (not shown) of the driving circuit board 123, so as to receive a driving signal, such as a driving frequency and a driving voltage. Through this structure, a circuit is formed by the piezoelectric pin 1223d, the piezoelectric carrying plate 1223a, the adjusting resonance plate 1223b, the piezoelectric plate 1223c, the conducting electrode 1225b, the conductive frame 1225 and the conducting pin 1225a for transmitting the driving signal. Moreover, the insulation frame 1224 is insulated between the conductive frame 1225 and the actuator element 1223, so as to avoid the occurrence of a short circuit. Thereby, the driving signal is transmitted to the piezoelectric plate 1223c. After receiving the driving signal such as the driving frequency and the driving voltage, the piezoelectric plate 1223c deforms due to the piezoelectric effect, and the piezoelectric carrying plate 1223a and the adjusting resonance plate 1223b are further driven to generate the bending deformation in the reciprocating manner.

Furthermore, in the embodiment, the adjusting resonance plate 1223b is located between the piezoelectric plate 1223c and the piezoelectric carrying plate 1223a and served as a cushion between the piezoelectric plate 1223c and the piezoelectric carrying plate 1223a. Thereby, the vibration frequency of the piezoelectric carrying plate 1223a is adjustable. Basically, the thickness of the adjusting resonance plate 1223b is greater than the thickness of the piezoelectric carrying plate 1223a, and the vibration frequency of the actuator element 1223 can be adjusted by adjusting the thickness of the adjusting resonance plate 1223b.

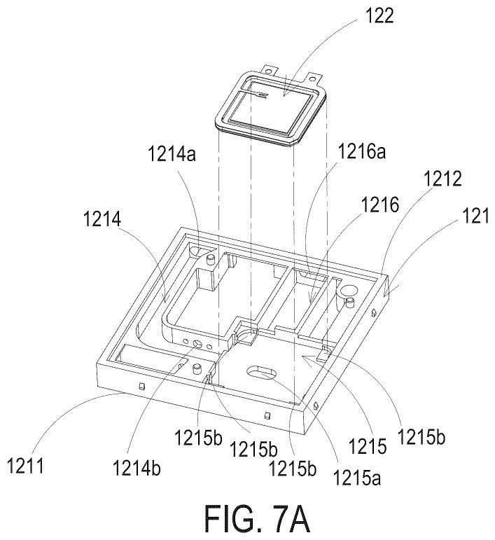

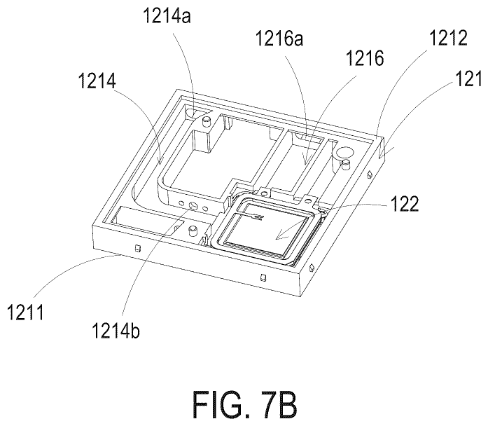

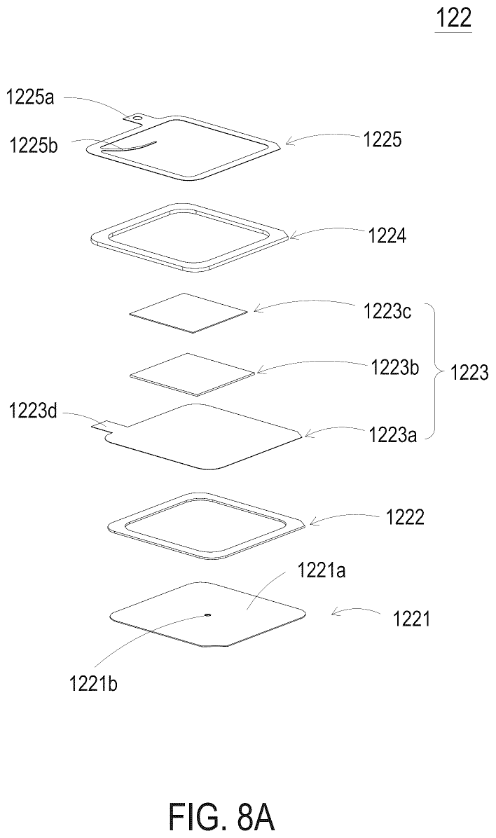

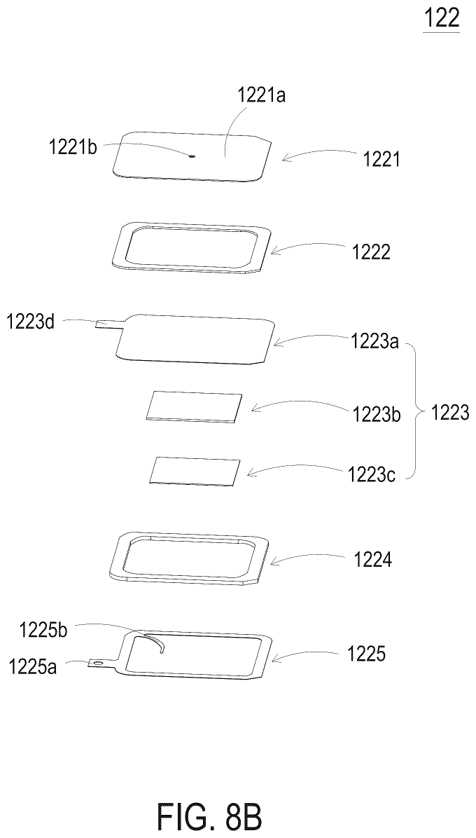

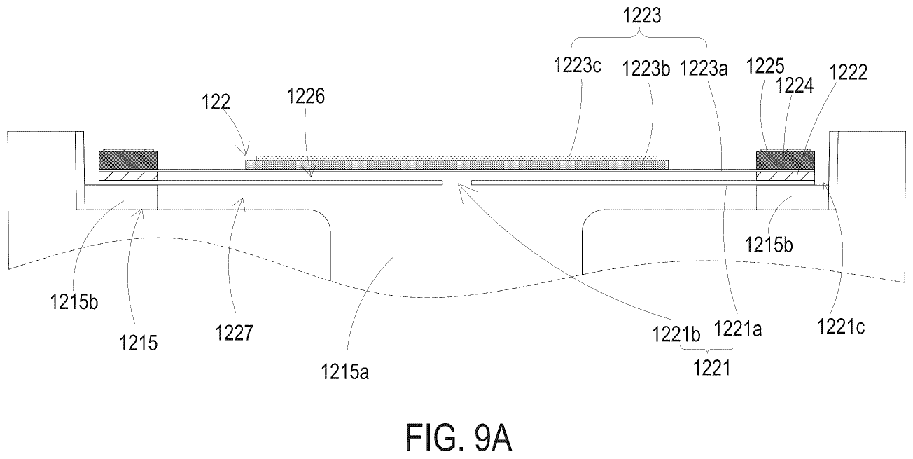

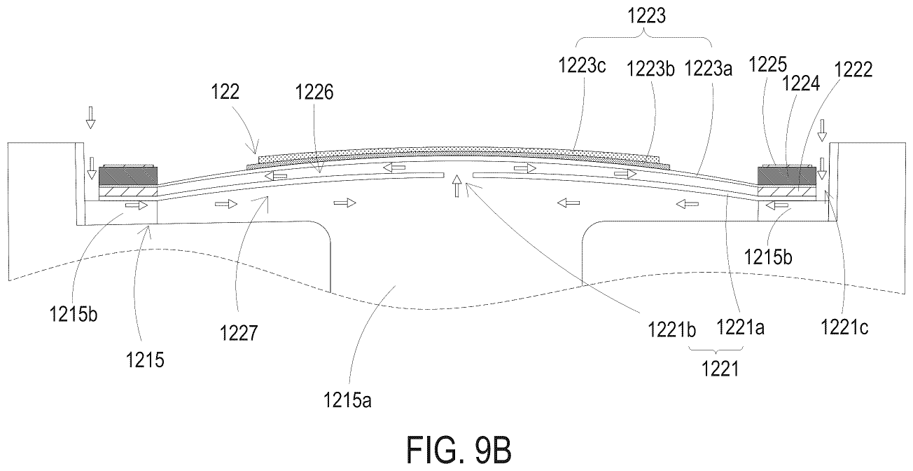

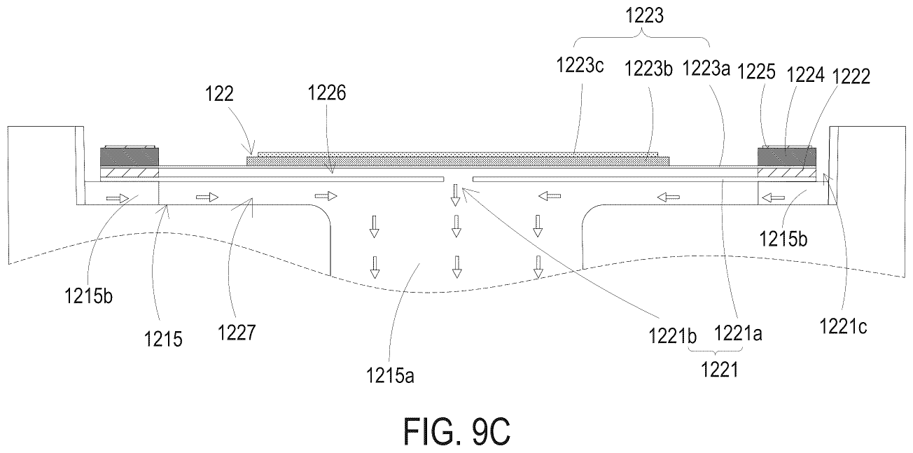

Please further refer to FIG. 7A, FIG. 7B, FIG. 8A, FIG. 8B and FIG. 9A. In the embodiment, the gas-injection plate 1221, the chamber frame 1222, the actuator element 1223, the insulation frame 1224 and the conductive frame 1225 are stacked and positioned in the gas-guiding-component loading region 1215 sequentially, so that the piezoelectric actuator 122 is supported and positioned in the gas-guiding-component loading region 1215. A plurality of clearances 1221c are defined between the suspension plate 1221a of the gas-injection plate 1221 and an inner edge of the gas-guiding-component loading region 1215 for gas flowing therethrough. In the embodiment, a flowing chamber 1227 is formed between the gas-injection plate 1221 and the bottom surface of the gas-guiding-component loading region 1215. The flowing chamber 1227 is in communication with the resonance chamber 1226 between the actuator element 1223, the chamber frame 1222 and the suspension plate 1221a through the hollow aperture 1221b of the gas-injection plate 1221. By controlling the vibration frequency of the gas in the resonance chamber 1226 to be close to the vibration frequency of the suspension plate 1221a, the Helmholtz resonance effect is generated between the resonance chamber 1226 and the suspension plate 1221a, so as to improve the efficiency of gas transportation. When the piezoelectric plate 1223c is moved away from the bottom surface of the gas-guiding-component loading region 1215, the suspension plate 1221a of the gas-injection plate 1221 is driven to move away from the bottom surface of the gas-guiding-component loading region 1215 by the piezoelectric plate 1223c. In that, the volume of the flowing chamber 1227 is expanded rapidly, the internal pressure of the flowing chamber 1227 is decreased to form a negative pressure, and the gas outside the piezoelectric actuator 122 is inhaled through the clearances 1221c and enters the resonance chamber 1226 through the hollow aperture 1221b. Consequently, the pressure in the resonance chamber 1226 is increased to generate a pressure gradient. When the suspension plate 1221a of the gas-injection plate 1221 is driven by the piezoelectric plate 1223c to move toward the bottom surface of the gas-guiding-component loading region 1215, the gas in the resonance chamber 1226 is discharged out rapidly through the hollow aperture 1221b, and the gas in the flowing chamber 1227 is compressed, thereby the converged gas is quickly and massively ejected out of the flowing chamber 1227 under the condition close to an ideal gas state of the Benulli's law, and transported to the ventilation hole 1215a of the gas-guiding-component loading region 1215.

By repeating the above operation steps shown in FIG. 9B and FIG. 9C, the piezoelectric plate 1223c is driven to generate the bending deformation in a reciprocating manner. According to the principle of inertia, since the gas pressure inside the resonance chamber 1226 is lower than the equilibrium gas pressure after the converged gas is ejected out, the gas is introduced into the resonance chamber 1226 again. Moreover, the vibration frequency of the gas in the resonance chamber 1226 is controlled to be close to the vibration frequency of the piezoelectric plate 1223c, so as to generate the Helmholtz resonance effect to achieve the gas transportation at high speed and in large quantities. The gas is inhaled through the gas-inlet 1214a on the outer cover 126, flows into the gas-inlet groove 1214 of the base 121 through the gas-inlet 1214a, and is transported to the position of the particulate sensor 125. The piezoelectric actuator 122 is enabled continuously to inhale the gas into the inlet path, and facilitate the gas outside the gas detection module to be introduced rapidly, flow stably, and transported above the particulate sensor 125. At this time, a projecting light beam emitted from the laser component 124 passes through the transparent window 1214b to irritate the suspended particles contained in the gas flowing above the particulate sensor 125 in the gas-inlet groove 1214. When the suspended particles contained in the gas are irradiated and generate scattered light spots, the scattered light spots are received and calculated by the particulate sensor 125 for obtaining related information about the sizes and the concentration of the suspended particles contained in the gas. Moreover, the gas above the particulate sensor 125 is continuously driven and transported by the piezoelectric actuator 122, flows into the ventilation hole 1215a of the gas-guiding-component loading region 1215, and is transported to the gas-outlet groove 1216. At last, after the gas flows into the gas outlet groove 1216, the gas is continuously transported into the gas-outlet groove 1216 by the piezoelectric actuator 122, and thus the gas in the gas-outlet groove 1216 is pushed to discharge through the gas-outlet 1216a and the outlet opening 1261b.

The gas detector 1 of the present disclosure not only can detect the particulate matters in the gas, but also can detect the gas characteristics of the introduced gas, for example, to determine whether the gas is formaldehyde, ammonia, carbon monoxide, carbon dioxide, oxygen, ozone, or the like. Therefore, in one or some embodiments, the gas detector 1 of the present disclosure further includes a gas sensor 127 positioned and disposed on the driving circuit board 123, electrically connected to the driving circuit board 123, and accommodated in the gas-outlet groove 1216, so as to detect the air pollution introduced into the gas-outlet groove 1216. Preferably but not exclusively, in an embodiment, the gas sensor 127 includes a volatile-organic-compound sensor for detecting the information of carbon dioxide (CO₂) or volatile organic compounds (TVOC). Preferably but not exclusively, in an embodiment, the gas sensor 127 includes a formaldehyde sensor for detecting the information of formaldehyde (HCHO) gas. Preferably but not exclusively, in an embodiment, the gas sensor 127 includes a bacteria sensor for detecting the information of bacteria or fungi. Preferably but not exclusively, in an embodiment, the gas sensor 127 includes a virus sensor for detecting the information of virus in the gas. Preferably but not exclusively, the gas sensor 127 is a temperature and humidity sensor for detecting the temperature and humidity information of the gas.

Please refer to FIG. 2H. The air guiding fan 21 of the present disclosure is controlled to start and guide the air pollution to pass through the filter component 22 for filtration, and the filter component 22 includes HEPA, UVC, activated carbon, photocatalysis, plasma, negative ion and other filtering and purification technologies. Preferably but not exclusively, the filter component 22 is a filter with a grade of minimum filtration efficiency value (MREV) 8 or above, or a filter with high-efficiency particulate air (HEPA) grade, which is configured to absorb the chemical smoke, the bacteria, the dust particles and the pollen contained in the air pollution, so that the air pollution introduced into the filter component 22 is filtered and purified to achieve the effect of filtering and purification. Notably, in the present disclosure, the filter with the high efficiency particulate air (HEPA) grade is of 10 or above, and has a dust holding capacity greater than 12,000 mg. In an embodiment, the filter component 22 is a filter with a ultra-low penetrating air (ULPA) grade of 14 so as to improve the filtration efficiency and meet the higher cleanliness requirements. In an embodiment, the filter component 22 of the present disclosure is further combined with physical or chemical materials to provide a sterilization effect on the air pollution, and the airflow of the air guiding fan 21 flows in the path indicated by the arrow. In an embodiment, the filter component 22 includes a decomposition layer coated thereon to sterilize in chemical means. Preferably but not exclusively, the decomposition layer includes an activated carbon 22a configured to remove organic and inorganic substances in air pollution, and remove colored and odorous substances. Preferably but not exclusively, the activated carbon has a formaldehyde absorption capacity greater than 1,500 mg. In an embodiment, the decomposition layer includes a cleansing factor containing chlorine dioxide layer 22b configured to inhibit viruses, bacteria, fungi, influenza A, influenza B, enterovirus and norovirus in the air pollution, and the inhibition ratio can reach 99% and more, thereby reducing the cross-infection of viruses. In an embodiment, the decomposition layer includes an herbal protective layer 22c extracted from ginkgo and Japanese Rhus chinensis configured to resist allergy effectively and destroy a surface protein of influenza virus (such as H1N1 influenza virus) passing therethrough. In an embodiment, the decomposition layer includes a silver ion 22d configured to inhibit viruses, bacteria and fungi contained in the air pollution. In an embodiment, the decomposition layer includes a zeolite 22e configured to remove ammonia nitrogen, heavy metals, organic pollutants, Escherichia coli, phenol, chloroform and anionic surfactants. Furthermore, in some embodiments, the filter component 22 is combined with a light irradiation element to sterilize in chemical means. Preferably but not exclusively, the light irradiation element is a photo-catalyst unit including a photo catalyst 22f and an ultraviolet lamp 22g. When the photo catalyst 22f is irradiated by the ultraviolet lamp 22g, the light energy is converted into the chemical energy, thereby decomposes harmful gases and disinfects bacteria contained in the air pollution, so as to achieve the effects of filtering and purifying. Notably, the power of the ultraviolet lamp 22g in the present disclosure is more than 120 mw. In an embodiment, the light irradiation element is a photo-plasma unit including a nanometer irradiation tube 22h. When the introduced air pollution is irradiated by the nanometer irradiation tube 22h, the oxygen molecules and water molecules contained in the air pollution are decomposed into high oxidizing photo-plasma, and an ion flow capable of destroying organic molecules is generated. In that, volatile formaldehyde, volatile toluene and volatile organic compounds (VOC) contained in the air pollution are decomposed into water and carbon dioxide, so as to achieve the effects of filtering and purifying. Moreover, in some embodiments, the filter component 22 is combined with a decomposition unit to sterilize in chemical means. Preferably but not exclusively, the decomposition unit is a negative ion unit 22i with a dust collecting plate. It makes the suspended particles in the air pollution to carry with positive charge and adhered to the dust collecting plate carry with negative charges, so as to achieve the effects of filtering and purifying. Preferably but not exclusively, the decomposition unit is a plasma ion unit 22j. The oxygen molecules and water molecules contained in the air pollution are decomposed into positive hydrogen ions (H⁺) and negative oxygen ions (O²⁻) by the plasma ion. The substances attached with water around the ions are adhered on the surface of viruses and bacteria and converted into OH radicals with extremely strong oxidizing power, thereby removing hydrogen (H) from the protein on the surface of viruses and bacteria, and thus decomposing (oxidizing) the protein, so as to filter the introduced air pollution and achieve the effects of filtering and purifying.

In summary, the present disclosure provides an indoor air pollution-free cleanroom system that achieves artificial intelligence green and healthy building standards. An intelligently controlled indoor air pollution detection and purification system and an advanced indoor and outdoor air pollution treatment device are integrated with an efficient building design structure, a renewable energy system, a water resource management system, a circular economy and resource recycling, and combined with a net zero carbon emission goal and an efficient air purification technology, and it allows to achieve low energy consumption and high comfort requirements in green building standards (LEED standards) and healthy building standards (WELL standards), and achieve new artificial intelligence green and healthy building standards, thereby controlling the indoor air pollution to be completely clean, the water pollution to zero, the noise pollution to zero and the net carbon emissions to be zero, and maximizing the energy saving. It allows to achieve carbon neutrality while keeping indoor air free of pollutants. Consequently, the cleanliness of the cleanroom level is achieved, and the health and comfort of the living environment is improved.

What is claimed is:

1. An indoor air pollution-free cleanroom system achieving artificial intelligence green and healthy building standards, comprising:
an indoor air pollution detection and purification system, comprising a plurality of gas detectors, at least one indoor air pollution treatment device, at least one networked cloud computing service device and at least one storage and data processing device, wherein the plurality of gas detectors comprehensively monitor indoor air pollution, the indoor air pollution treatment device filters and purifies the indoor air pollution, an intelligent networking system of the networked cloud computing service device and an artificial intelligence generated content (AIGC) model are used to perform dynamic monitoring and adjustment analysis and a control instruction is issued based on analysis results, to adjust an operating mode of the indoor air pollution treatment device;
a high-efficiency building design structure, comprising a passive design structure and an airtight design structure, wherein the passive design structure comprises a natural ventilation structure, a natural lighting structure and a thermal insulation structure, wherein the airtight design structure is used to improve a building airtightness;
a renewable energy system, comprising a solar photovoltaic system, a wind energy system, a geothermal energy system and an energy storage system for providing electricity to a building and storing excess electricity;
a water resource management system, comprising a rainwater collection system and a high-efficiency water-saving device, wherein the rainwater collection system is used to recycle rainwater, wherein the high-efficiency water-saving device comprises a low-flow toilet and an intelligent sprinkler system configured to use rainwater recycled by the rainwater collection system; and
a circular economy and resource recycling, comprising a building material recycling and a construction resource recycling, wherein the building material recycling is configured to recycle building demolition or renovation materials, and the construction resource recycling is configured to promote an efficient use of resources within a building life cycle.

2. The indoor air pollution-free cleanroom system achieving artificial intelligence green and healthy building standards according to claim 1, wherein the indoor air pollution treatment device of the indoor air pollution detection and purification system is disposed in an indoor field, and comprises at least one of the plurality of gas detectors, at least one air guiding fan, at least one filter component and at least one driving controller disposed therein, wherein the gas detector and the driving controller are electrically connected, and the gas detector receives the control instruction through Internet of Things communication to control actuation operation of the air guiding fan, for carrying out circulating air pollution purification and completely clean room treatment in the indoor field.

3. The indoor air pollution-free cleanroom system achieving artificial intelligence green and healthy building standards according to claim 2, wherein the indoor air pollution treatment device comprises a gas exchange device, a purifier, a fan filter unit (FFU), an exhaust device, a cooling and heating device, a range hood, a humidity control device or a mobile vacuum cleaner.

4. The indoor air pollution-free cleanroom system achieving artificial intelligence green and healthy building standards according to claim 1, wherein the networked cloud computing service device comprises a wireless network cloud computing service module, a cloud control service unit, a device management unit, an application unit and the artificial intelligence generated content (AIGC) model, the storage and data processing device collects information data to store and form a big data database, and the artificial intelligence generated content (AIGC) model captures the big data database through calculation, comparison and recognition to generate automatically-generated data.

5. The indoor air pollution-free cleanroom system achieving artificial intelligence green and healthy building standards according to claim 1, wherein the generative artificial intelligence (AIGC) model comprises technology of a big data database, a big data analysis, a computing power calculation, a transportation capacity allocation, an early warning system, an air pollution flow algorithm model, a field demand equivalent, an equipment configuration, an equipment intelligent control, an intelligent energy management system, an integration of the system to form AI data calculation global intelligent control, and an integration of the indoor air pollution treatment device to achieve automatic control.

6. The indoor air pollution-free cleanroom system achieving artificial intelligence green and healthy building standards according to claim 3, wherein the indoor air pollution detection and purification system comprises a central control computer intelligent control device, the central control computer intelligent control device receives the control instruction issued by the networked cloud computing service device through Internet of Things communication, and transmits the control instruction received to the gas detector of the indoor air pollution treatment device through Internet of Things communication to control the actuation operation of the air guiding fan, wherein the central control computer intelligent control device comprises an edge computing function and allows receiving and analyzing air quality data monitored by each of the gas detectors of the indoor air pollution treatment device through Internet of Things communication, the control instruction is generated based on analysis results and directly issued, and the control instruction is transmitted to a corresponding one of the gas detectors of the indoor air pollution treatment device through Internet of Things communication, and received by the corresponding one of the gas detectors, whereby the actuation operation of the air guiding fan is controlled, and automatic control and optimization of the indoor air pollution treatment device is achieved.

7. The indoor air pollution-free cleanroom system achieving artificial intelligence green and healthy building standards according to claim 3, wherein the filter component is a filter with a minimum efficiency reporting value (MREV) of 8 or above.

8. The indoor air pollution-free cleanroom system achieving artificial intelligence green and healthy building standards according to claim 3, wherein the filter component is a filter with a high efficiency particulate air (HEPA) grade.

9. The indoor air pollution-free cleanroom system achieving artificial intelligence green and healthy building standards according to claim 8, wherein the filter with the high efficiency particulate air (HEPA) grade is of 10 or above, and has a dust holding capacity greater than 12,000 mg.

10. The indoor air pollution-free cleanroom system achieving artificial intelligence green and healthy building standards according to claim 3, wherein the filter component is a filter with a ultra-low penetrating air (ULPA) grade of 14.

11. The indoor air pollution-free cleanroom system achieving artificial intelligence green and healthy building standards according to claim 3, wherein the filter component is combined with a decomposition layer coated thereon, and the decomposition layer uses chemical means to sterilize the air pollution, wherein the decomposition layer comprises an activated carbon, and the activated carbon has a formaldehyde absorption capacity greater than 1,500 mg.

12. The indoor air pollution-free cleanroom system achieving artificial intelligence green and healthy building standards according to claim 3, wherein the filter component is combined with a light irradiation element to sterilize in chemical means.

13. The indoor air pollution-free cleanroom system achieving artificial intelligence green and healthy building standards according to claim 12, wherein the light irradiation element is a photo-catalyst unit including a photo catalyst and an ultraviolet lamp, and the ultraviolet lamp has a power more than 120 mw.

14. The indoor air pollution-free cleanroom system achieving artificial intelligence green and healthy building standards according to claim 12, wherein the light irradiation element is a photo-plasma unit including a nanometer irradiation tube.

15. The indoor air pollution-free cleanroom system achieving artificial intelligence green and healthy building standards according to claim 3, wherein the filter component is combined with a decomposition unit to sterilize in chemical means.

16. The indoor air pollution-free cleanroom system achieving artificial intelligence green and healthy building standards according to claim 15, wherein the decomposition unit is a negative ion unit or a plasma ion unit.

17. The indoor air pollution-free cleanroom system achieving artificial intelligence green and healthy building standards according to claim 2, wherein Internet of Things communication is a wireless communication for communicating with the networked cloud computing service device via a wireless connection, wherein the wireless communication transmission comprises one selected from the group consisting of a Wi-Fi module, a Bluetooth module, a radio frequency identification module and a near field communication (NFC) module.

18. The indoor air pollution-free cleanroom system achieving artificial intelligence green and healthy building standards according to claim 2, wherein Internet of Things communication is a wired communication for communicating with the networked cloud computing service device via a wired line.

19. The indoor air pollution-free cleanroom system achieving artificial intelligence green and healthy building standards according to claim 2, wherein the gas detector comprises a controlling circuit board, a gas detection main part, a microprocessor and a communicator, the controlling circuit board is electrically connected to the driving controller, and the gas detection main part, the microprocessor and the communicator are integrally packaged on the controlling circuit board and electrically connected to the controlling circuit board, wherein the microprocessor controls the detection of the gas detection main part, the gas detection main part detects air pollution, and the microprocessor processes the air pollution to output and provide air pollution information to the communicator for a communication transmission externally.

20. The indoor air pollution-free cleanroom system achieving artificial intelligence green and healthy building standards according to claim 1, wherein the indoor air pollution-free cleanroom system achieves a cleanliness of cleanroom level at ZAPClean Room 1˜12.
